(19) United States
(12) Patent Application Publication
(10) Pub. No.: US 2009/0311403 A1
(43) Pub. Date: Dec. 17, 2009

(54) Method of Using Organic Compounds

(75) Inventor: Willi Grab, Orpund (CH)

Correspondence Address: NORRIS, MCLAUGHLIN & MARCUS, 875 THIRD AVE, 18TH FLOOR, NEW YORK, NY, 10022, US

(73) Assignee: Givaudan SA, Vemier (CH)

(21) Appl. No.: 12/375,298

(22) PCT Filed: Jul. 24, 2007
(86) PCT No.: PCT/CH07/00365

§ 371 Date: Jan. 27, 2009

(30) Foreign Application Priority Data
Jul. 28, 2006 (SG) 200605098-3

(51) Int. Cl. A23L 1/226; C07D 405/04; C07D 317/30; C07D 319/06

(52) U.S. Cl. 426/535; 544/336; 549/454; 549/372; 426/536; 426/537

(57) ABSTRACT

A method of using organic compounds and providing slow release flavoring in or for food products wherein flavor precursors are added to flavor compositions and/or food products and release flavor compounds upon consumption of the food products, and novel flavor precursor compounds. The flavor precursors can be prepared from monoglycerides and flavor compounds which comprise one or more carbonyl groups.

The present invention relates to flavor precursors that can be added to food to slowly release the flavor upon heating and consumption.

Food products are rendered more palatable and more attractive for consumers by adding various flavors to them. However, when food products are processed or stored for a longer period of time, many flavors get lost at least partially, in particular the volatile flavors. For this reason, many attempts at encapsulation of flavors (binding to matrixes or coating to prevent premature release) have been made to retard the flavor release and prolong shelf life. These however are only partially successful, and there remains a need of a method or flavor product that keeps the main part of volatile flavors inside the food product to be slowly released upon consumption of the food by the consumer.

Applicant has now found that the use of flavor precursors, according to formula I (monoglyceride acetals and ketals) below in flavor compositions and food products provides a means to retard the premature release of volatile flavors and slowly release them upon consumption. Thereby an early unwanted flavor release, for example during storage or processing, is minimised, the shelf life of the flavored food product is prolonged and the flavor of the food product is improved.

A flavor precursor compound according to formula I

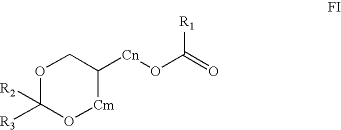

wherein n and m are selected from 0 and 1, and if n is 1 then m is 0, and if n is 0 then m is 1;

wherein R1 is selected from the group consisting of

a straight-chain C7 to C17 alkyl, a straight-chain C7 to C17 alkenyl, a branched C7 to C17 alkyl, a branched C7 to C17 alkenyl,

a straight-chain C7 to C17 monoalkenyl, a branched C7 to C17 monoalkenyl,

a straight-chain C7 to C17 alkadienyl, a branched C7 to C17 alkadienyl,

a straight-chain C7 to C17 alkatrienyl, a branched C7 to C17 alkatrienyl,

a straight-chain C9 to C17 alkatetraenyl, a branched C9 to C17 alkatetraenyl,

and a straight-chain C11 to C17 alkapentaenyl, a branched C11 to C17 alkapentaenyl, and

wherein the R2-CO-R3 part of formula I is the residue of a flavor compound, wherein the O of the R2-CO-R3 is the remainder of the reacted carbonyl group of said flavor compound,

and wherein R2-CO-R3 has a molecular weight from 44 to 294.

Certain alpha monoglyceride acetals/ketals of Fl with m=0 and n=1, and their synthesis, have been described in JP2001181271; these are useful as intermediates for the synthesis of monoglycerides, which are used as emulsifiers or moisturizers in cosmetics.

### SUMMARY OF THE INVENTION

In a first aspect, there is provided a method of providing a flavored food product, wherein at least one flavor precursor of formula I

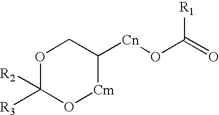

wherein n and m are selected from 0 and 1, and if n is 1 then m is 0, and if n is 0 then m is 1;

wherein R1 is selected from the group consisting of

a straight-chain C7 to C17 alkyl, a straight-chain C7 to C17 alkenyl, a branched C7 to C17 alkyl, a branched C7 to C17 alkenyl,

a straight-chain C7 to C17 monoalkenyl, a branched C7 to C17 monoalkenyl,

a straight-chain C7 to C17 alkadienyl, a branched C7 to C17 alkadienyl,

a straight-chain C7 to C17 alkatrienyl, a branched C7 to C17 alkatrienyl,

a straight-chain C9 to C17 alkatetraenyl, a branched C9 to C17 alkatetraenyl,

and a straight-chain C11 to C17 alkapentaenyl, a branched C11 to C17 alkapentaenyl, and

wherein the R2-CO-R3 part of formula I is the residue of a flavor compound, wherein the O of the R2-CO-R3 is the remainder of the reacted carbonyl group of said flavor compound,

and wherein R2-CO-R3 has a molecular weight from 44 to 294;

is admixed to a food product in a sufficient concentration to release a flavor of noticeable aroma upon consumption and/or heating of the food product.

In another aspect, there is provided a flavor composition comprising at least one flavor precursor defined as described herein-above.

In another aspect, there is provided a flavor composition comprising a mixture of flavor precursors as defined herein formed by reacting at least one flavor compound comprising one or more carbonyl group with at least one monoglyceride in an acid catalyzed reaction.

In another aspect, there is provided a food product comprising at least one flavor precursor defined as described herein-above.

In another aspect, there is provided a food product comprising a mixture of flavor precursors as defined in any one of claims 1 to 8 formed by reacting at least one flavor compound comprising one or more carbonyl group with at least one monoglyceride in an acid catalyzed reaction.

In another aspect, there is provided at least one flavor pecursor compound defined as described herein-above wherein m=1 and n=0.

In another aspect, there is provided a process of producing the flavor precursor of claim 11 by reacting at least one flavor compound comprising one or more carbonyl group with at least one monoglyceride in an acid catalyzed reaction.

### DETAILED DESCRIPTION OF THE INVENTION

In one embodiment, there is provided a method as described herein-above wherein said flavor is selected from the group consisting of flavors as described herein-below.

In another embodiment, there is provided a method as described herein-above wherein

R2 is selected from the group consisting of H, a C1 to C15 alkyl, a C1 to C15 oxoalkyl, a C1 to C15 hydroxyalkyl, C2 to C15 alkenyl, a C2 to C15 oxoalkenyl, and a C2 to C15 hydroxyalkenyl,

R3 is selected from the group consisting of C1 to C15 straight-chain alkyl comprising one or two substituents independently selected from O, OH, N, NH, phenyl, furanyl, C9 aryl, C10 aryl, SH, and SR4, wherein R4 is a straight-chain or branched C1 to C5 alkyl residue optionally selected from methyl, ethyl, propyl, isopropyl, butyl, isobutyl, pentyl and isopentyl, and the total number of carbon atoms in R3 and R4 is up to 15, C1 to C15 straight-chain alkyl, comprising one or two atoms independently selected from O,S or N within the alkyl chain, C3 to C15 singly, doubly or multiply branched alkyl optionally substituted with one or two residues independently selected from O, OH, N, NH, SH, and SR4, wherein R4 is a straight-chain or branched C1 to C5 alkyl residue optionally selected from methyl, ethyl, propyl, isopropyl, butyl, isobutyl, pentyl and isopentyl, and the total number of carbon atoms in R3 and R4 is up to 15,

C3 to C15 singly, doubly or multiply branched alkyl comprising one or two atoms independently selected from O,S or N within the alkyl chain, C2 to C15 straight-chain alkenyl, C3 to C15 straight-chain alkadienyl,

C2 to C15 straight-chain alkenyl optionally substituted with one or more residues independently selected from O, OH, N, NH, phenyl, furanyl, C9 aryl, C10 aryl, SH, SR4 wherein R4 is a straight-chain or branched C1 to C5 alkenyl residue optionally selected from ethenyl, propenyl, butenyl, and pentenyl, and the total number of carbon atoms in R3 and R4 is up to 15,

C2 to C15 straight-chain alkenyl, optionally comprising one or more atoms independently selected from O,S and N within the alkyl chain,

C3 to C15 branched alkenyl, optionally substituted with one or two alkyl groups,

C2 to C15 branched alkenyl comprising one or two alkyl groups optionally substituted with one or two residues independently selected from O, OH, N, NH, phenyl, furanyl, C9 aryl, C10 aryl, SH, SR4 wherein R4 is a straight-chain or branched C1 to C5 alkenyl residue optionally selected from ethenyl, propenyl, butenyl, and pentenyl, and the total number of carbon atoms in R3 and R4 is up to 15,

C3 to C15 branched alkenyl comprising one or two alkyl groups and one or more atoms independently selected from O,S and N within the alkyl chain,

C5 to C15 singly, doubly, or multiply branched alkadienyl, optionally substituted with one or two alkyl groups,

C4 to C15 singly, doubly, or multiply branched alkadienyl comprising one or more atoms independently selected from O,S and N within the alkenyl chain;

a 5- or 6-membered carbon ring residue comprising up to two heteroatoms independently selected from O, S, and N, optionally substituted with one or more residues independently selected from O, OH, alkoxy, alkyl, alkenyl;

a C1 to C6 alkyl or alkenyl substituted with a 5- or 6-membered carbon ring residue comprising up to two heteroatoms independently selected from O, S, and N, optionally substituted with one or more residues independently selected from O, OH, alkoxy, alkyl, alkenyl;

a R2-C-R3 ring residue selected from oxacyclopentan optionally substituted with a residue selected from methyl, dimethyl, ethyl, hydroxy, and methoxy, oxacyclopentene optionally substituted with a residue selected from methyl, dimethyl, ethyl, hydroxy, and methoxy, hydroxycyclopentene, hydroxyalkylcyclopentene, hydroxymethylcyclopentene, hydroxydimethylcyclopentene, ethylhydroxycyclopentene, and ethylhydroxymethylcyclopentene, thiacyclopentane, alkylthiacyclopentane, and alkyl-3-thiacyclopentane, oxacyclopentene, 2-oxacyclopentene, 3-oxacyclopentene, alkyloxacylcopentene, alkyl-3-oxacyclopentene, methyl-3-oxa-cyclopentene, alkyl-3-oxacyclopentene, dimethyl-3-oxacyclopentene, ethyl-3-oxacyclopentene, ethylmethyl-3-oxacyclopentene, hydroxyalkyl-3-oxacyclopentene, hydroxymethyl-3-oxacyclopentene, hydroxydimethyl-3-oxacyclopentene, hydroxyethyl-3-oxacyclopentene, ethylhydroxymethyl-3-oxacyclopentene, oxacyclopentane, 3-oxacyclopentan, alkyl-3-oxacyclopentan, methyl-3-oxacyclopentan, dimethyl-3-oxacyclopentan, ethyl-3-oxacyclopentan, ethylmethyl alkyl-3-oxacyclopentan, alkylcyclopenten, alkenylcyclopenten, alkylalkenylcyclopenten, methylcylopenten, dimethylcyclopenten, ethylmethylcyclopenten, propylmethylcyclopenten, butylmethylcyclopenten, butenylmethylcyclopenten, pentylmethylcyclopenten, pentenylmethylcyclopenten, alkylcarboxy-alkenyl-cyclopentan, alkylcarboxy-alkyl-cyclopentan, methylcarboxy-alkenyl-cyclopentan, methylcarboxy-alkyl-cyclopentan, alkylcarboxy-pentenyl-cyclopentan, alkylcarboxy-pentyl-cyclopentan, methylcarboxy-alkenyl-cyclopentan, and methylcarboxy-alkyl-cyclopentan, and wherein the alkyl is selected from methyl, ethyl, propyl, butyl, pentyl, pentenyl, hexyl, heptyl, and octyl, oxo-alkylcyclohexen, oxo-methylcyclohexen, oxo-trimethylcyclohexen, oxo-ethylcyclohexen, oxo-ethylmethylcyclohexen, oxo-propylcyclohexen, oxo-propylmethylcyclohexen, oxo-isopropylcyclohexen, oxo-isopropylmethylcyclohexen, oxo-thiopropylcyclohexen, oxo-thiopropylmethylcyclohexen, alkylcyclohexen, methylcyclohexen, trimethylcyclohexen, ethylcyclohexen, ethylmethylcyclohexen, propylcyclohexen, propylmethylcyclohexen, isopropylcyclohexen, isopropylmethylcyclohexen, thiopropylcyclohexen, thiopropylmethylcyclohexen, alkylcyclohexan, methylcyclohexan, trimethylcyclohexan, ethylcyclohexan, ethylmethylcyclohexan, propylcyclohexan, propylmethylcyclohexan, isopropylcyclohexan, isopropylmethylcyclohexan, thiopropylcyclohexan, thiopropylmethylcyclohexan;

and wherein R2 and R3 together have a total number of carbon atoms of up to 15, and the R2-C-R3 has a total number of carbon atoms of up to 16.

In another embodiment, there is provided a method as described herein-above wherein R1 is an alkyl selected from the group consisting of C7, C8, C9, C10, C11, C13, C15, and C17 alkyl.

In another embodiment, there is provided a method as described herein-above wherein R1 is an alkenyl selected from the group consisting of C7, C8, C9, C10, C11, C13, C15, and C17 alkenyl, a C17-8en (oleic acid residue) alkenyl, a C17-8,11 alka-dienyl (linoleic acid residue), and a C17-8,11,14-trienyl (linolenic acid residue).

In another embodiment, there is provided a method as described herein-above wherein R2 is selected from the group consisting of H, methyl and ethyl.

In another embodiment, there is provided a method as described herein-above wherein R3 is selected from the group consisting of a C1 to C8 straight-chain alkyl, a C1 to C8 branched alkyl comprising up to two alkyl groups, wherein the alkyl residue may contain one or more further alkyl residues, a C1 to C8 straight-chain alkyl comprising one or two substituents selected from O, and SR4, wherein R4 is an alkyl residue selected from methyl, and ethyl, a C2 to C8 straight-chain alkenyl, and a ring selected from a 5 membered and a 6 membered ring, comprising up to two N heteroatoms, wherein the ring may be further substituted with one or more alkyl groups selected from methyl, ethyl, propyl and isopropyl.

In another embodiment, there is provided a method as described herein-above wherein R1 is an alkyl selected from the group consisting of C7, C9, C11, C13, C15 and C17 alkyl, R2 is selected from the group consisting of H, methyl and ethyl, and R3 is selected from the group consisting of a C1 to C8 straight-chain alkyl, a C1 to C8 branched alkyl comprising one or two alkyl groups, wherein the alkyl residue may contain one or more further alkyl residues, a C1 to C8 straight-chain alkyl comprising one or two substituents selected from O, and SR4, wherein R4 is an alkyl residue selected from methyl, and ethyl, a C2 to C8 straight-chain alkenyl, and a ring selected from a 5 membered and 6 membered ring comprising up to two N heteroatoms, wherein the ring may be further substituted with one or more alkyl groups selected from methyl, ethyl, propyl and isopropyl.

The flavor precursors of formula I are acetals (for R2=H) or ketals (for R2=optionally substituted alkyl or alkenyl) of formula I shown herein-below. They slowly release volatile flavors (aroma) and thereby protect the chemically bound flavor from an undesired excessive release of the volatile flavor compound, for example during processing and storage of a food product. The flavor is slowly released in presence of water and enzymes that are present naturally in the mouth during consumption.

Flavor precursors for use in the present invention can be formed by reaction of flavor compounds comprising one or more carbonyl group with monoglycerides (1-acylglycerides and 2-acylglycerides) in an acid-catalyzed reaction (compare reaction scheme below).

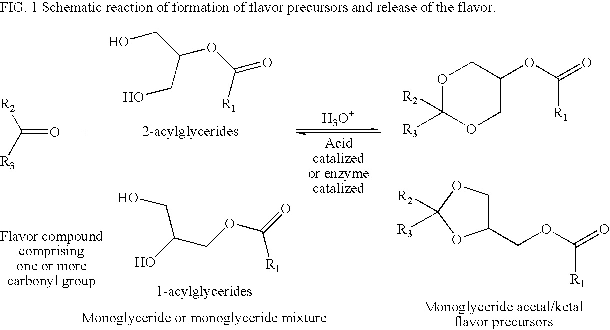

The compounds in the upper part show a 2-acylglyceride and its product, the compounds in the bottom part show the 1-acylglyceride and its product. Formation of the flavor precursors occurs in the presence of H+, see left to right direction of the scheme.

These flavor precursors will then slowly release the flavor (R2-CO-R3) in a reversed reaction when exposed to aqueous acidic conditions (compare right to left direction of the scheme), for example upon heating in the presence of water, for example by cooking or by baking and in particular in the mouth which contains enzymes that speed up the flavor release.

The reacted and later released flavors comprise the residues R2 and R3 and a carbonyl group (R2-CO-R3). For example, a precursor of formula I wherein R2=Hydrogen and R3=pent-2-en-1-yl will release 3-hexenal, a precursor of formula I wherein R2=methyl and R3=1-oxobutyl will release 2,3-hexandione, a precursor of formula I wherein R2=H and R3=2-methylthioethyl will release methional.

Monoglycerides can be prepared from natural sources including plant and animal sources by enzymatic or chemical hydrolysis, as is well-known in the art. Some monoglycerides are available commercially as a mixture or in pure form (either extracted or synthesized). For example, without limitation, plant sources of monoglycerides include palm oil, oil seeds, sunflower seeds, nuts, cacoa beans, coconuts, hazelnuts, peanuts, and many more. For example, without limitation, animal sources of monoglycerides include milk, butter, meat, chicken, beef, porc, lamb, fish, and many more.

A typical mixture of monoglycerides derived from natural sources includes monoglycerides bound to saturated fatty acids including laurylmonoglyceride (C12—forms Fl with R1=C11), myristylmonoglyceride (C14—forms Fl with R1=C13), palmitylmonoglyceride (C16—forms Fl with R1=C15), and stearoylmonoglyceride (C18—forms Fl with R1=C17). Monoglycerides comprising unsaturated fatty acids include oleylmonoglyceride, linolylmonoglyceride, and alpha-linolenylmonoglyeride.

A typical mixture of monoglycerides derived from cocoa butter includes palmitylmonoglyceride (C16), oleylmonoglyceride (C18-1, with one double bond), and stearoylmonoglyceride (C18).

A typical mixture of monoglycerides derived from miglyol, a commonly used oil from vegetable sources), includes C8, C10, and C12 monoglycerides.

A typical mixture of monoglycerides derived from butter includes mainly C8, C10, C12, C14, C16, C18 saturated monoglycerides.

When used in food, monoglycerides or their residues (R1 in formula I) can partially degrade over time and release free fatty acid, which may turn a product rancid. When choosing a particular precursor for a particular food product, R1 of said percursor will be chosen so that upon degradation it releases e.g. caprylic acid that contribute to the desired flavor note rather than, for example, lauric acid, that will result in an undesirable soapy off-note in the chosen food product. The specific choice of appropriate R1 is well within the experience of the skilled person and depends on the food product and desired flavor note.

Commercial monoglycerides are mixtures of mainly 1-acylglyceddes with variable amounts of 2-acylglycerides and other glycerides. 1-acylglycerides when reacted with flavor compounds as described herein will form flavor precursors of formula I wherein n=1 and m=0. 2-acylglycerides when reacted with flavor compounds as described herein will form flavor precursors of formula I wherein n=0 and m=1.

Flavors are compounds that can be detected by the human olfactory system. To provide sensory properties, a flavor must have the following molecular properties: some water solubility, a sufficiently high vapor pressure, low polarity, and some ability to dissolve in fat (lipophilicity). Flavor compounds have a molecular weight of up to 294 (no larger compounds are known to trigger the human olfactory system).

Flavors useful for the methods described herein are compounds that comprise one or more carbonyl group and that can be reacted with the monoglycerides as described herein. These flavors include but are not limited to aldehydes, ketones, and other flavor classes provided they comprise one or more carbonyl groups.

Useful flavors include natural and artificial flavors, and extracts from natural sources that contain a mixture of flavor compounds, and flavor compounds as such. A number of suitable flavors can be found, for example, in the BACIS database (Boelens Aroma Chemical Information Service), which includes the Flavor-Base 2004 database (Leffingwell & Associates, Canton, Ga., USA), in the listing of Flavor chemicals on the FDA (Food & Drug Administration, USA) & FEMA GRAS lists (FEMA—Flavor and Extracts Manufacturers Association, GRAS—Generally Recognised As Safe), and the European Community (EC) Register list.

Examples of useful flavor compounds include, but are not limited to (+)-8,9-DEHYDRONOOTKATONE; (1R-CIS AND TRANS)-2-(1-ACETYLTHIO-1-METHYL)ETHYL-5-METHYLCYCLOHEXANONE; (E)-2-(2-OCTENYL)CYCLOPENTANONE; (E)-5-ISOPROPOXY-2-DECENAL; (E,E)-3,5-OCTADIEN-2-ONE; (METHYLTHIO)ACETONE; (RAC)-3-ACETYLOXY-5-METHYL-2-HEXANONE; 1-(2,5,5-TRIMETHYL-CYCLOPENT-1-EN-1-YL)-3-METHYL-2-BUTEN-1-ONE; 1-(3,5,5,-TRIMETHYL-1-CYCLOHEXENYL)-3-METHYL-2-BUTEN-1-ONE; 1(5-METHYLFURYL-2)-PROPANE-1,2-DIONE; 1-(METHYLTHIO)-2-BUTANONE; 1-(METHYLTHIO)-3-PENTANONE; 1-(METHYLTHIO)-OCTAN-3-ONE; 1-(P-METHOXYPHENYL)-2-PROPANONE; 1,4-DODEC-6-ENOLACTONE; 1,5,5-TRIMETHYL-8-ETHYL-G-OXA-BICYCLO(4,3,0)NON-6-en-3-one; 1,5-[Z]-OCTADIEN-3-ONE; 10-UNDECENAL; 12-METHYL-TRIDECANAL; 14-METHYL-PENTADECANAL; 1-HYDROXY-2-BUTANONE; 1-MERCAPTO-2-PROPANONE; 1-MERCAPTO-3-PENTANONE; 1-PENTEN-3-ONE; 2-(3,3-DIMETHYLCYCLOHEXYLIDEN)-ETHANAL; 2-(METHYLTHIO)METHYL-2-BUTENAL; 2-(METHYLTHIOMETHYL)-3-PHENYLPROPENAL; 2,10-UNDECADIENAL; 2,3-DIHYDRO-2,3,3-TRIMETHYL-1H-INDEN-1-ONE; 2,4-DIMETHYL-HEPT-4-ENE-3-ONE; 2,4-PENTADIENAL; 2,5-DIETHYL-4-HYDROXY-5-METHYL-3(2H)-FURANONE; 2,5-DIMETHYL-3(2H)-FURANONE; 2,5-DIMETHYL-4-HYDROXY-6-HEPTANONE; 2,5-DIMETHYL-TETRAHYDRO-3-FURANONE; 2,6-DIMETHYL-3-HYDROXY-4-PYRONE; 2,6-DIMETHYL-4-HEPTANONE; 2,6-DIMETHYLBICYCLO[3.2.1]OCT-2-EN-7-ONE; 2-AMINO-ACETOPHENONE; 2-BUTYL-2-BUTENAL; 2-ETHYL-4-METHYL-PENT-2-ENAL; 2-HEPTEN-4-ONE; 2-HEPTYL-BUTYROLACTONE; 2-Hexenal; 2-HYDROXY-1-(4-HYDROXY-3-METHOXY-PHENYL)-ETHANONE; 2-HYDROXY-2-CYCLOHEXEN-1-ONE; 2-HYDROXY-3,4,5-TRIMETHYL-2-CYCLOPENTEN-1-ONE; 2-HYDROXY-3,5,5-TRIMETHYL-2-CYCLOHEXENONE; 2-HYDROXYACETOPHENONE; 2-ISOPROPYL-5-CAPROLACTONE; 2-ISOPROPYL-DIOXOLAN-4-ONE; 2-METHOXY-3,4,5-TRIMETHYL-2-CYCLOPENTEN-1-ONE; 2-METHYL-2-OCTENAL; 2-METHYL-3-(P-METHYLPHENYL)PROPANAL; 2-METHYL-8-PHENYL-OCT-2-ENE-6-ONE; 2-METHYLCYCLOHEXANONE; 2-METHYLHEPTAN-3-ONE; 2-METHYLHEXAN-3-ONE; 2-METHYLNONANAL; 2-METHYL-SPIRO(5,5)-UNDECAN-1-ONE; 2-OCTEN-4-ONE; 2-PHENYL-3-(2-FURYL)PROP-2-ENAL; 2-PHENYL-4-PENTENAL; 2-TETRADECENAL; 2-TRANS-4-CIS-7-CIS-TRIDECATRIENAL; 3-((2-METHYL-3-FURYL)THIO)-4-HEPTANONE; 3(2)-HYDROXY-4-METHYL-HEXAN-2(3)-ONE; 3-(3-METHOXY-PHENYL)-PROPAN-2-ONE; 3-(5-METHYL-2-FURYL)BUTANAL; 3(Z),6(Z)-9(Z)-DODECATRIENAL; 3(Z),6(Z)-DODECADIENAL; 3,3-DIETHOXY-2-BUTANONE; 3,3-DIMETHYLCYCLOHEXANONE; 3,4,5,6-TETRAHYDRO PSEUDO IONONE; 3,4-DIMETHYL-BUTYROLACTONE; 3,5-DIMETHYL-4-THIA-HEPTAN-2,6-DIONE; 3,6,DIMETHYL-5,6-DIHYDRO-2(4H)BENZOFURANONE; 3,6-DIMETHYL-2-HYDROXY-2-CYCLOHEXEN-1-ONE; 3,7-DIMETHYL-6-OCTEN-2-ONE; 3A,4,5,7A-TETRAHYDRO-3,6-DIMETHYL-2(3H)BENZOFURANONE; 3-ACETYLOXY-2-OCTANONE; 3-ACETYLOXY-2-PENTANONE; 3-ACETYLSULFANYL-2-ETHYLHEXANAL; 3-DECANONE; 3-ETHOXY HEXANAL; 3-ETHYL-2-HYDROXY-4-METHYLCYCLOPENT-2-EN-1-ONE; 3-ETHYL-4,5,5-TRIMETHYL-2-(5H)-FURANONE; 3-HEPTYLDIHYDRO-5-METHYL-2(3H)-FURANONE; 3-HEXANONE; 3-HYDROXY-2-PENTANONE; 3-HYDROXY-4,5,5-TRIMETHYL-2-(5H)-FURANONE; 3-HYDROXY-4-METHYL-5-PENTYL-2(5H)-FURANONE; 3-HYDROXY-4-METHYL-ALPHA-PYRONE; 2-ACETYL-4,5-DIMETHYL-THIAZOLE; 2-METHYL-3-ACETYLPYRIDINE; 1-ETHYL-2-ACETYL-PYRROLE; 1-METHYL-2-ACETYLPYRROLE; 2,4-DIMETHYL-5-ACETYL THIAZOLE; 3-ACETYL-2,5-DIMETHYLFURAN; 1,4-DIMETHYL-4-ACETYL-1-CYCLOHEXENE; 4-ACETYL-6-T-BUTYL-1,1-DIMETHYLINDAN; 4-ACETYL-2-METHYLPYRIMIDINE; ACETYL-2-PYRAZINE; ACETYL-2-FURAN; ACETYL-2-PYRROLE; ACETYL-2-PYRIDINE; ACETYL-2-DIMETHYL-PYRAZINE; ACETYL-2-THIAZOLE; ACETYL-3 PYRIDIN; ACETYL-3-DIMETHYL-2.5-THIOPHENE; ACETYL-2 METHYL-5 FURAN; ACETYL-2-THIAZOLINE-2; ACETYL-2 METHYL-3 PYRAZINE; PROPIONYL PYRAZINE; PROPIONYL-2-METHYL-3-FURANE; 2-PROPIONYLTHIAZOLE; 2-PROPIONYLPYRROLE; 3-HYDROXY-4-PHENYL-2-BUTANONE; 3-HYDROXY-5-HEXYL-4-METHYL-2(5H)-FURANONE; 3-HYDROXY-6-HYDROXYMETHYL-PYRAN-2-ONE; 3-HYDROXYMETHYL-2-OCTANONE; 3-ISOAMYL-2,4-PENTANDIONE; 3-MERCAPTO-2-METHYLPENTANAL; 3-MERCAPTO-2-OCTANONE; 3-MERCAPTO-2-PENTANONE; 3-MERCAPTO-4-PHENYL-2-BUTANONE; 3-MERCAPTO-5-METHYL-HEXAN-2-ONE; 3-METHYL NON-2(E)-EN-40NE; 3-METHYL-1-CYCLOPENATADECANONE; 3-METHYL-2-CYCLOHEXEN-1-ONE; 3-METHYL-2-CYCLOPENTEN-1-ONE; 3-METHYL-2-HEPTEN-4,5-DIONE; 3-METHYL-3-(3-METHYL-BUT-2-ENYL)-DIHYDRO-FURAN-2-ONE; 3-METHYL-4-PHENYL-3-BUTENE-2-ONE; 3-METHYL-5-PROPYL-2-CYCLOHEXEN-1-ONE; 3-METHYLCYCLOHEXANONE; 3-METHYLENE-2-OCTANONE; 3-METHYL-GAMMA-DECALACTONE; 3-METHYLPENTANAL; 3-METHYLTHIOHEXANAL; 3-NONANONE; 3-NONEN-2-ONE; 3-PENTANONE; 3-PENTEN-2-ONE; 3-PENTYL-4,5,5-TRIMETHYL-2-(5H)-FURANONE; 3-PHENYL-4-PENTENAL; 3-PROPYL-2-CYCLOPENTEN-1-ONE; 3-PROPYLIDEN-HEPTAN-2-ONE; 4-(2,6,6-TRIMETHYL-2-CYCLOHEXEN-1-YL)BUTAN-2-ONE; 4-(2,6,6-TRIMETHYL-CYCLOHEXA-1,3-DIENYL)-BUT-3-EN-2-ONE; 4-(FURAN-2-YL)-BUTAN-2-ONE; 4-(METHYLTHIO)BUTANAL; 4-(P-TOLYL)-2-BUTANONE; 4,4-DIMETHOXY-3-HEXANONE; 4,4-DIMETHYL-1,3-OXATHIANE-2-ONE; 4,8-DIMETHYL-3,7-NONADIEN-2-ONE; 4-[(2-FURANMETHYL)THIO]-2-PENTANONE; 4-ACETOXY-HEX-4-EN-3-ONE; 4-EPOXY-1,1,3-TRIMETHYLCYCLOHEX-5-EN-2-ONE; 4-ETHYL-1,3-OXATHIAN-2-ONE; 4-ETHYL-5-METHYL-1,3-OXATHIAN-2-ONE; 4-HEPTYL-3-METHYLBUTYROLACTONE; 4-HYDROXY-2,2,5-TRIMETHYL-3[2H]-FURANONE; 4-HYDROXY-3,5,5-TRIMETHYL-CYCLOHEX-5,5-EN-1-ONE; 4-HYDROXY-4-METHYL-5-HEXENOIC-ACID LACTONE; 4-HYDROXY-4-METHYL-CIS-7-DECANOICACID-4-LACTONE; 4-MERCAPTO-4-METHYL-PENTAN-2-ONE; 4-METHYL-2-PENTENAL; 4-METHYL-3-PENTEN-2-ONE; 4-METHYL-4-FURFURYLTHIO-2-PENTANONE; 4-METHYL-5-PENTYL-DIHYDRO-2(3H)-FURANONE; 4-METHYLCYCLOHEXANONE; 4-METHYLTHIO-2-PENTANONE; 4-OXO-BETA DAMASCONE; 4-PHENYL-3,5-DITHIA-2-HEXANONE; 5 ALPHA-ANDROST-16-EN-3-ONE; 5,5-DIMETHYL-2(5H)-FURANONE; 5,5-DIMETHYL-2-METHOXY-CYCLOHEX-2-ENONE; 5-ETHYL-2-HYDROXY-3,4-DIMETHYL-2-CYCLOPENTEN-1-ONE; 5-ETHYL-2-METHOXY-3,4-DIMETHYL-2-CYCLOPENTEN-1-ONE; 5-ETHYLDIHYDRO-5-METHYL-2(3H)-FURANONE; 5-HYDROXY-4-OCTANONE; 5-HYDROXY-8-UNDECENOIC ACID D-LACTONE; 5-METHYL-2-PROPYL-[1,3]-DIOXOLAN-4-ONE; 5-METHYL-3-(3-METHYL-BUT-2-ENYL)-DIHYDRO-FURAN-2-ONE; 5-METHYL-3-HEXEN-2-ONE; 5-METHYL-4-HEXENE-2,3-DIONE; 5-METHYL-5-HEXEN-2-ONE; 5-PENTYL-ALPHA-PYRONE; 5-P-TOLUYL-2(3H)-FURANONE; 6-(1-PENTENYL)-2H-PYRAN-2-ONE; 6-HEPT-1-ENYL-5,6-DIHYDRO-PYRAN-2-ONE; 6-HYDROXY-3,7-DIMETHYLOCTANOIC ACID LACTONE; 6-METHYL-3-HEPTEN-2-ONE; 6-METHYL-NON-5-EN-4-ONE; 6-METHYL-OCTANAL; 6-PROPYL-[1,3]OXATHIAN-2-ONE; 6-UNDECANONE; 7-METHYL-2,3,4,4A,5,6-HEXAHYDRO-2-NAPHTHALENONE; 7-METHYL-6-OCTEN-2-ONE; 7-OCTENAL; 8-(2,2-DIMETHYLCYCLOPROPYL)-OCTA-3,5-DIEN-2-ONE; 8A-METHYL-3,4,4A,5,8,8A-HEXAHYDRO-1(2H)-NAPHTALENONE; 8-METHYL NONANAL; 8-METHYL-TRIDEC-7-EN-6-ONE; 8-NONEN-2-ONE; 8-NONENAL; 9-DECENAL; ACETONE; ACETOPHENONE; A-P-DIMETHYLANISALACETONE; AR-TURMERONE; BENZOPHENONE; BENZOYLACETONE; BENZYL DIPROPYL KETONE; BENZYL-ACETONE; BENZYLIDENE ACETONE; BETA-IRONE; BICYCLO-IRONE; BICYCLONE; BICYCLONONALACTONE; BUTYRO-1,4-LACTONE; CARYOPHYLLEN-12-AL; CIS,CIS-4,7-DECADIEN-1-AL; CIS-2-NONENAL; CIS-3-HEXENAL; CIS-3-NONENAL; CIS-4-HEPTEN-2-ONE; CIS-4-HEXENAL; CIS-4-NONENAL; CIS-5-NONENAL; CIS-5-OCTENAL; CIS-7-NONENAL; CITRONELLENE LACTONE; CLONAL; COGNAC LACTONE; CYCLOBUTONE; CYCLOHEPTADECA-9-EN-1-ONE; CYCLOHEXANONE; CYCLOIONONE; CYCLOPENTANONE; CYLCOHEPTADECANONE; D-1-(2,6,6-TRIMETHYL-3-CYCLOHEXEN-1-YL)-2-BUTEN-1-ONE; DAMASCENONE; DECANAL; DECENYL CYCLOPENTANONE; DIBENZYL KETONE; DIBUTYL-4,4 GAMMA BUTYROLACTONE; DIHYDRO FILBERTONE; DIHYDRO-5-METHYL-5-PROPYL-2(3H)-FURANONE; DIHYDROCARVONE; DIHYDRO-DEHYDRO-GAMMA-IONONE; DIHYDROJASMONE LACTONE; DIHYDROLAVENDELLACTONE; DIMETHYL HYDROQUINONE; DIMETHYL METHOXY FURANONE; DIMETHYL-2,4 ACETOPHENONE; DIMETHYL-2,6 OCTANAL; DIMETHYL-3,6-BENZO-2(3H)-FURANONE; DODEC-2-EN-1,5-LACTONE; DODEC-2-EN-4-ONE; DODECANAL; E-DECALACTONE; E-DODECALACTONE; ETHYL-2-HEPTEN-2-AL; ETHYL-3 CYCLOPENTANDIONE; FARNESYL ACETONE; FURFURYLIDENE ACETONE; GAMMA DAMASCONE; GAMMA IONONE; GERANYL ACETONE; GINGERONE; HEPT-3-EN-2-ONE; HEPTANAL; HEXANAL; HEXEN 5 ONE; HEXENYL METHYL KETONE; HEXYLIDEN CYCLOPENTANONE; HOMOCORYLONE; HYDROBICYCLON E; HYDROXY-3(2)-HEPTAN-2(3)-ONE; HYDROXY-5-DECADIENOIC ACID-2,4 LACTONE; ISO JASMONE; ISOLONGIFOLENE-KETONE; ISOPHORONE; JASMIN LACTONE; LAVENDELLACTONE; MASSOIA LACTONE; MELONAL, DIMETHYL HEPTENAL; MENTHONE; METHIONAL; METHOXY-P-PHENYL-PENTEN-3-ONE, ETHONE; METHYL 2 OCTANAL; METHYL 2-PENTENE-1-AL; METHYL THIO-METHYLTHIOMETHYL-2-PENTENAL; METHYL-2 UNDECANAL; METHYL-2-BUTANAL; METHYL-2-PENT-2-ENAL; METHYL-2-TETRAHYDRO-FURANONE; METHYL-4-HYDROXY-3/2H/-FURANONE; METHYL-5-HEPT-2-EN-4-ONE; METHYL-6-HEPTA-3,5-DIENONE; METHYLTHIO-3-BUTANAL; METHYLTHIO-4-BUTAN-2-ONE; MINTLACTONE; MYRTENAL; NEOFOLIONE; NEOHESPERIDINE DIHYDROCHALCONE; NONANAL; NOOTKATONE; OCTAN-2-ONE; OCTANAL; PENNYROYAL OIL 84% PULEGONE; PENTADECANONE; PENTANAL; PENTYL 2-FURYL KETONE; PHENYL-2-BUTEN-2-AL; PHENYL-3-PROPANAL; PIPERITENONE; PIPERONYL ACETONE; P-MENTH-1-ENAL; P-MENTH-1-ENE-9-AL; P-MENTHAN-2-ONE; PRENAL; PROPANAL; PROPYL-ISO PARA ACETOPHENONE; RAC-3-HYDROXY-5-METHYL-2-(5H)-FURANONE; RASPBERRY KETONE; SAFRANAL; SAGE OIL 27% THUJONE; SOLANONE; SPIRO-(6,5)-DODECAN-1-ONE; TETRAHYDROIRONE; TETRAHYDRONOOTKATONE; TRANS-4-HEXENAL; TRANS-5-NONENAL; TRANS-6-DECEN-1-AL; TRANS-6-NONENAL; TRANS-7-METHYL-3-OCTEN-2-ONE; TRANS-7-NONENAL; TRIDECAN-2-ONE; TRIMETHYL-3,5,5-HEXANAL; TRITHIOACETONE; UNDECANAL; VANILLYLIDENE ACETONE; and VERBENONE.

Useful flavor precursors according to formula I are, without limitation, the following groups of compounds below with a particular selection of R1, R2, R3 and a R2-C-R3 ring residue (N and m are as defined in Fl, either n=1 and m=0 or n=0 and m=1).

Where branched compounds are indicated, these may be singly, doubly or multiply branched (having one, two or more alkyl groups).

Without Limitation, Useful Groups of Compounds are as Follows:

R1 and R2 According to Formula I:

Compounds of formula I with R3 selected as indicated in the table below.

R1and R3 According to Formula I:

Compounds of formula I with R2 selected from H, methyl, ethyl, butyl, 2-butenyl, and vinyl.

R2 and R3 According to Formula I:

Compounds of formula I with R1 selected from C7 to C17 alkyl, C7 to C17 alkenyl, C7 to C17 monoalkenyl and C7 to C17 alkadienyl.

Compounds of formula I with R1 selected from straight-chain C7 to C17 alkyl, straight-chain C7 to C17 alkenyl, straight chain C7 to C17 monoalkenyl, and straight-chain C7 to C17 alkadienyl.

Compounds of formula I with R1=alkenyl selected from C7, C8, C9, C10, C11, C13, C15, and C17 alkenyl, C17-8en alkenyl (oleic acid residue), C17-8,11 alka-dienyl (linoleic acid residue), and C17-8,11,14-trienyl (linolenic acid residue).

Compounds of formula I with R1=straight-chain alkenyl selected from C7, C8, C9, C10, C11, C13, C15, and straight-chain C17 alkenyl, straight-chain C17-8en alkenyl (oleic acid residue), straight-chain C17-8,11 alka-dienyl (linoleic acid residue), and C17-8,11,14-trienyl (linolenic acid residue).

Compounds of formula I with R1=heptyl, nonyl, undecyl, tridecyl, pentadecyl, heptadecyl.

R1 According to Formula I:

Compounds of formula I with R2 selected from H, methyl, ethyl, 2-butenyl, or vinyl, and R3 as indicated in the table below.

Compounds of formula I with R2=H, and R3 selected as indicated in the table below.

Compounds of formula I with R2=methyl, and R3 selected as indicated in the table below.

R2 According to Formula I:

Compounds of formula I with R1 selected from C7 to C17 alkyl, C7 to C17 alkenyl, C7 to C17 monoalkenyl and C7 to C17 alkadienyl, and R3 selected as indicated in the table below.

Compounds of formula I with R1 selected from heptyl, nonyl, undecyl, tridecyl, pentadecyl, heptadecyl, and R3 selected as indicated in the table below.

R3 According to Formula I:

Compounds of formula I with R1 selected from heptyl, nonyl, undecyl, tridecyl, pentadecyl, and heptadecyl, and R2 selected from H, methyl, ethyl, 2-butenyl, and vinyl.

Compounds with R1, R2 and R3 (Table) as Defined Below:

Compounds of formula I with R1 selected from C7 to C17 alkyl, C7 to C17 alkenyl, C7 to C17 monoalkenyl and C7 to C17 alkadienyl, and R2 selected from H, methyl, ethyl, butyl, 2-butenyl, or vinyl, and R3 selected as indicated in the table below.

Compounds of formula I with R1=heptyl, nonyl, undecyl, tridecyl, pentadecyl, and heptadecyl, wherein R2 is H, and R3 is selected as indicated in the table below.

Compounds of formula I with R1 selected from heptyl, nonyl, undecyl, tridecyl, pentadecyl, and heptadecyl, R2=methyl, and R3 selected as indicated in the table below.

TABLE 1
Examples groups of R3 residues of formula I and the groups of
compounds defined herein-above.
R3 =

C3 to C15 branched alkyl comprising one or two alkyl groups, wherein
the alkyl residue optionally contains one or more further alkyl residues
C1 to C15 straight-chain alkyl comprising one or two substituents
selected from O, OH, N, NH, SH, and SR4, wherein R4 is an alkyl
residue selected from methyl, ethyl, propyl, isopropyl, butyl, isobutyl,
pentyl and isopentyl and the total number of carbon atoms in R3
and R4 is up to 15,
C3 to C15 branched alkyl comprising one or two alkyl groups and
substituted with one or two residues independently selected from O, OH,
N, NH, SH, and SR4, wherein R4 is an alkyl group selected from methyl,
ethyl, propyl, isopropyl, butyl, isobutyl, pentyl and isopentyl, and the total
number of carbon atoms in R3 and R4 is up to 15
C1 to C15 straight-chain alkyl, comprising one or two atoms
independently selected from O, S and N within the alkyl chain
C3 to C15 branched alkyl comprising one or two alkyl groups and
comprising one or two atoms independently selected from O, S and N
within the alkyl chain
C2 to C15 straight-chain alkenyl,
C3 to C15 straight-chain alkadienyl
C3 to C15 branched alkenyl, substituted with one or two alkyl groups,
C5 to C15 branched alkadienyl, substituted with one or two alkyl
groups
C2 to C15 straight-chain alkenyl comprising one or two substituents
selected from O, OH, N, NH, SH, SR4 wherein R4 is an alkenyl residue
selected from ethenyl, propenyl, butenyl, and pentenyl, and the total
number of carbon atoms in R3 and R4 is up to 15,
C2 to C15 branched alkenyl comprising one or two alkyl groups
comprising one or two substituents selected from O, OH, N, NH,
SH, and SR4, wherein R4 is a alkenyl residue selected from ethenyl,
propenyl, butenyl, and pentenyl,
and the total number of carbon atoms in
R3 and R4 is up to 15
C2 to C15 straight-chain alkenyl, comprising one or two atoms
independently selected from O, S and N within the alkyl chain
C3 to C15 branched alkenyl comprising one or two alkyl groups and
comprising one or two atoms independently selected from O, S and
N within the alkyl chain
C4 to C15 branched alkadienyl, comprising one or two alkyl groups and
comprising one or two atoms independently selected from O, S and N
within the alkyl chain
Straight-chain C1 to C15 alkyl, straight-chain C2 to C15 alkenyl, branched
C1 to C15 alkyl, branched C3 to C15 alkenyl
C2 to C5 alkyl substituted in position 1 with a substituent selected from O
and OH
C2 to C13 alkyl
C2 to C10 alkyl
C3 to C13 alkenyl
C2 to C10 alkenyl
compounds as defined in FI wherein R3 has a maximum of up to 13
carbon atoms
straight-chain C1 to C13 alkyl
straight-chain C1 to C10 alkyl
straight-chain C2 to C13 alkenyl
straight-chain C2 to C10 alkenyl
branched C1 to C13 alkyl comprising one or two branching alkyl groups
branched C1 to C10 alkyl comprising one or two branching alkyl groups
singly or doubly branched C4 to C13 alkenyl comprising one or two
branching alkyl groups
singly or doubly branched C4 to C10 alkenyl comprising one or two
branching alkyl groups
C3 to C13 alkenyl selected from 1-alkenyl, a 3-alkenyl, and a
1,3-alkadienyl
C3 to C10 alkenyl selected from 1-alkenyl, a 3-alkenyl, and a
1,3-alkadienyl
C3 to C13 alkenyl selected from propenyl, butenyl, pentenyl, hexenyl,
heptenyl, and octenyl
C3 to C10 alkenyl selected from propenyl, butenyl, pentenyl, hexenyl,
heptenyl, and octenyl
singly, doubly, or multiply branched C3 to C13 alkenyl
singly, doubly or multiply branched C3 to C10 alkenyl
4-methylpent-3-enyl or 4-methylpent-1,3-dienyl
methylpentenyl and methylpentadienyl
straight-chain C3 to C13 alkenyl selected from propenyl, butenyl,
pentenyl, hexenyl, heptenyl, and octenyl
straight-chain C3 to C10 alkenyl selected from propenyl, butenyl,
pentenyl, hexenyl, heptenyl, and octenyl
prop-1-enyl, but-1-enyl, pent-1-enyl, hex-1-enyl, hept-1-enyl, and
oct-1-enyl
pent-1,3-dienyl, hex-1,3-dienyl, hept-1,3-dienyl, and oct-1,3-dienyl
1-1-hydroxyethyl, 1-oxoethyl, 1-oxopropyl, 1-oxobutyl, 1-oxopentyl,
1-oxohexyl
C2 to C7 alkyl, C3 alkenyl
C3 alkenyl prop-1-enyl
prop-1-enyl
C3 alkenyl
straight-chain C2 to C7 alkyl
straight-chain C3 to C 15 alkenyl
straight-chain C3 to C 15 monoalkenyl
straight-chain 1-monoalkenyl
straight-chain C5 to C 13 alkadienyl
straight-chain 1,3-alkadienyl
straight-chain C8 to C 12 alkatrienyl
singly or doubly branched C3 to C 12 alkyl comprising one or two
branching alkyl groups
1-methyl alkyl and 3-methylalkyl
singly, doubly or multiply branched alkyl selected from 1-methyl alkyl,
3-methylalkyl, 1,3-methylalkyl, 1-ethylalkyl, 1,1-diethylalkyl, and
1-isopropylalkyl
3,6-methylalkyl
singly, doubly or multiply branched C3 to C13 monoalkenyl, singly,
doubly or multiply branched C3 to C13 alkadienyl and branched C3 to
C13 alkatrienyls
thioalkyl including thiomethyl, thioethyl, thiopropyl, thiobutyl, thiopentyl,
thiohexyl
methylthioalkyl including methylthiomethyl, methylthioethyl,
methylthiopropyl, methylthiobutyl, methylthiopentyl, methylthiohexyl
thioalkyl, thiomethyl, thioethyl, thiopropyl, thiobutyl, thiopentyl,
thiomethylethyl, thiomethylpropyl, thiomethylbutyl,
alkyl(methylthio)alkyl, methyl(methylthio)alkyl, methyl(methylthio)ethyl,
methyl(methylthio)propyl, methyl(methylthio)butyl
branched alkylthioalkyl including methyl-methylthioalkyl and
dimethylthioalkyl (including methyl-methylthiopropyl,
methyl-methylthiobutyl, methyl-methylthiopentyl, thiohexyl)
methylthiomonoalkenyl including methylthioethenyl, methylthiopropenyl,
methylthiobutenyl, methylthiopentenyl, thiohexenyl
thiaalkyl including thiamethyl, thiaethyl, thiapropyl, thiabutyl, thiapentyl,
thiahexyl
methylthiaalkyl including methylthiamethyl, methylthiaethyl,
methylthiapropyl, methylthiabutyl, methylthiapentyl, methyl-thiahexyl
singly, doubly or multiply branched alkylthiaalkyl including
methyl-methylthiaalkyl and dimethylthiaalkyl (including methylthiapropyl,
methylthiabutyl, methylthiapentyl, thiahexyl) (for example: 1,1 dimethyl
2,3 dithiabutyl)
methylthiamonoalkenyl including methylthiaethenyl, methylthiapropenyl,
methylthiabutenyl, methylthiapentenyl, thiahexenyl
dithiaalkyl including dithiamethyl, dithiaethyl, dithiapropyl, dithiabutyl,
dithiapentyl, dithiahexyl
methyldithiaalkyl including methyldithiamethyl, methyldithiaethyl,
methyldithiapropyl, methyldithiabutyl, methyldithiapentyl,
methyldithiahexyl
singly, doubly or multiply branched alkylthiaalkyl including
methylmethylthiaalkyl and dimethylthiaalkyl (including methylthiapropyl,
methylthiabutyl, methylthiapentyl, thiahexyl) (for example: 1,1 dimethyl
2,3 dithiabutyl)
methylthioalkenyl including methylthiomethenyl, methylthioethenyl,
methylthiopropenyl, methylthiobutenyl, methylthiopentenyl,
methylthiohexenyl
singly, doubly or multiply branched alkylthioalkenyl including
methyl-methylthioalkenyl and dimethylthioalkenyl (including
methylthiopropenyl, methylthiobutenyl, methylthiopentenyl, thiohexenyl)
methylthiomonoalkenyl including methylthioethenyl, methylthiopropenyl,
methylthiobutenyl, methylthiopentenyl, thiohexenyl
thiaalkenyl including thiaethenyl, thiapropenyl, thiabutenyl, thiapentenyl,
thiahexenyl
methylthiaalkenyl including methylthiaethenyl, methylthiapropenyl,
methylthiabutenyl, methylthiapentenyl, thiahexenyl
singly, doubly or multiply branched alkylthiaalkenyl including
methyl-methylthiaalkenyl and dimethylthiaalkenyl (including
methylthiapropenyl, methylthiabutenyl, methylthiapentenyl, thiahexenyl)
(for example: 1,1-dimethyl 2,3-dithiabutyl)
methylthiamonoalkenyl including methylthiapropenyl, methylthiabutenyl,
methylthiapentenyl, thiahexenyl
dithiaalkenyl including dithiaethenyl, dithiapropenyl, dithiabutenyl,
dithiapentenyl, dithiahexenyl
methyldithiaalkenyl including methyldithiaethenyl, methyldithiapropenyl,
methyldithiabutenyl, methyldithiapentenyl, methyldithiahexenyl
branched alkylthiaalkenyl including methyl-methylthiaalkenyl and
dimethylthiaalkenyl (including methylthiapropenyl, methylthiabutenyl,
methylthiapentenyl, thiahexenyl) (for example: 1,1 dimethyl
2,3 dithiabutenyl)
methylthiamonoalkenyl including methylthiaethenyl, methylthiapropenyl,
methylthiabutenyl, methylthiapentenyl, thiahexenyl
phenyl, alkylphenyl, and substituted phenyl with the substituent selected
from OH, and alkoxy
methylphenyl, ethylphenyl, propylphenyl, isopropylphenyl, butylphenyl,
isobutylphenyl, and pentylphenyl
substituted phenyl selected from hydroxyphenyl, methoxyphenyl, and
ethoxyphenyl
doubly substituted phenyl selected from hydroxy-alkoxyphenyl,
hydroxymethoxyphenyl, alkoxy-alkylphenyl, methoxy-alkylphenyl,
hydroxy-alkylphenyl, and hydroxymethoxyphenyl
furanyl, alkylfuranyl, methylfuranyl, ethylfuranly, propylfuranyl,
isopropylfuranyl, butylfuranyl, isobutylfuranyl
thiophenyl, alkylthiophenyl, methylthiophenyl, ethylthiophenyl,
dialkylthiophenyl, dimethylthiophenyl, ethylmethylthiophenyl
phenylalkyl and phenylalkenyl
phenylalkyl selected from benzyl, phenylethyl, phenylpropyl, phenylbutyl,
phenylpentyl, phenylhexyl, phenylheptyl
phenylalkenyl selected from phenylethenyl, phenylpropenyl,
phenylbutenyl, phenylpentenyl, phenylhexenyl, phenylheptenyl
alkylthioalkyl, alkylthioalkenyl, alkyldisulfanylalkyl,
alkyldisulfanylalkenyl, methylthioalkyl, methyl-methylthioalkyl,
methylthioalkenyl, methyl-methylthioalkenyl, methylthioalkyl,
methyl-methylthioalkyl, methylthioalkenyl, methyl-methylthioalkenyl,
methyldisulfanylalkyl, methyl-methyldisulfanylalkyl,
methyldisulfanylalkenyl, methyl-methyldisulfanylalkenyl
residue having a maximum number of C atoms of 12 and selected from
singly branched hydroxyalkyl, doubly branched hydroxyalkyl, multiply
branched hydroxyalkyl, hydroxydialkylalkyl, hydroxydimethylalkyl,
hydroxydimethylbutyl, hydroxydimethylpentyl, hydroxydimethylhexyl,
hydroxydimethylheptyl, hydroxydimethyloctyl, hydroxydimethylnonyl
furanyl-substituted straight-chain or branched alkyl including methyl,
ethyl, propyl, butyl, and pentyl, optionally substituted with one or
more of OH, O, and SH;
straight-chain or branched furanyl-substituted alkylthioalkyl,
furanyl-substituted methylthioalkyl
straight-chain or branched substituted alkyl substituted with
one or more of O, and OH; a straight-chain or branched substituted
alkyl substituted with one or more of O and OH wherein the alkyl is
selected from ethyl, propyl, butyl, pentyl, hexyl, heptyl, and octyl;
a straight-chain or branched (alkoxycarbonyl)alkyl; a straight-chain or
branched (alkoxycarbonyl)alkyl wherein the alkyl is selected from
ethyl, propyl, butyl, pentyl, hexyl, heptyl, and octyl
alkylphenyl, phenylalkyl, C9 arylalkyl, C10 arylalkyl, phenylalkenyl, C9
arylalkenyl, C10 arylalkenyl, methoxyphenyl, methoxyphenylalkyl,
methoxyphenylalkenyl, hydroxyphenylalkyl, hydroxyphenylalkenyl,
hydroxyphenylalkyl
a C1 to C6 alkyl or alkenyl substituted with a ring residue selected from
cyclohexyl, cyclohexenyl, cyclopentyl, cyclopentenyl,
oxacyclohexyl, oxacyclohexenyl, oxacyclopentyl, oxacyclopentenyl,
thiacyclohexyl, thiacyclohexenyl, thiacyclopentyl, thiacyclopentenyl,
azacyclohexyl, azacyclohexenyl, azacyclopentyl, azacyclopentenyl,
dioxacyclohexyl, dioxacyclohexenyl, dioxacyclopentyl,
dioxacyclopentenyl, dithiacyclohexyl, dithiacyclohexenyl,
dithiacyclopentyl, dithiacyclopentenyl, diazacyclohexyl,
diazacyclohexenyl, diazacyclopentyl, diazacyclopentenyl,
oxathiacyclohexyl, oxathiacyclohexenyl, oxathiacyclopentyl,
oxathiacyclopentenyl, oxathiacyclohexyl, oxathiacyclohexenyl,
oxathiacyclopentyl, oxathiacyclopentenyl, oxathiacyclohexyl,
oxathiacyclohexenyl, oxathiacyclopentyl, oxathiacyclopentenyl,
azaoxacyclohexyl, azaoxacyclohexenyl, azaoxacyclopentyl,
azaoxacyclopentenyl, azaoxacyclohexyl, azaoxacyclohexenyl,
azaoxacyclopentyl, azaoxacyclopentenyl, azaoxacyclohexyl,
azaoxacyclohexenyl, azaoxacyclopentyl, azaoxacyclopentenyl,
azathiacyclohexyl, azathiacyclohexenyl, azathia cyclopentyl,
azathia cyclopentenyl, azathia cyclohexyl, azathia cyclohexenyl,
azathia cyclopentyl, azathia cyclopentenyl, azathiacyclohexyl,
azathiacyclohexenyl, azathiacyclopentyl, azathiacyclopentenyl,
wherein the ring residue may optionally be further substituted with one
or more substituents selected independently from methyl, ethyl, propyl,
isopropyl, and a ketogroup.
a ring residue selected from
cyclohexyl, cyclohexenyl, cyclopentyl, cyclopentenyl,
oxacyclohexyl, oxacyclohexenyl, oxacyclopentyl, oxacyclopentenyl,
thiacyclohexyl, thiacyclohexenyl, thiacyclopentyl, thiacyclopentenyl,
azacyclohexyl, azacyclohexenyl, azacyclopentyl, azacyclopentenyl,
dioxacyclohexyl, dioxacyclohexenyl, dioxacyclopentyl,
dioxacyclopentenyl, dithiacyclohexyl, dithiacyclohexenyl,
dithiacyclopentyl, dithiacyclopentenyl, diazacyclohexyl,
diazacyclohexenyl, diazacyclopentyl, diazacyclopentenyl,
oxathiacyclohexyl, oxathiacyclohexenyl, oxathiacyclopentyl,
oxathiacyclopentenyl, oxathiacyclohexyl, oxathiacyclohexenyl,
oxathiacyclopentyl, oxathiacyclopentenyl, oxathiacyclohexyl,
oxathiacyclohexenyl, oxathiacyclopentyl, oxathiacyclopentenyl,
azaoxacyclohexyl, azaoxacyclohexenyl, azaoxacyclopentyl,
azaoxacyclopentenyl, azaoxacyclohexyl, azaoxacyclohexenyl,
azaoxacyclopentyl, azaoxacyclopentenyl, azaoxacyclohexyl,
azaoxacyclohexenyl, azaoxacyclopentyl, azaoxacyclopentenyl,
azathiacyclohexyl, azathiacyclohexenyl, azathia cyclopentyl,
azathia cyclopentenyl, azathia cyclohexyl, azathia cyclohexenyl,
azathia cyclopentyl, azathia cyclopentenyl, azathiacyclohexyl,
azathiacyclohexenyl, azathiacyclopentyl, azathiacyclopentenyl,
wherein the ring residue may optionally be further substituted
with one or more substituents selected independently from methyl,
ethyl, propyl, isopropyl, and a ketogroup.
a ring residue selected from cyclohexyl, cyclohexenyl, cyclopentyl,
cyclopentenyl wherein the ring is substituted with at least one
ketogroup and further substituted with an alkyl selected from methyl
and ethyl.
a ring residue selected from thiacyclohexyl, thiacyclohexenyl,
thiacyclopentyl, thiacyclopentenyl wherein the ring is substituted
with a ketogroup, and wherein the ring may optionally be further
substituted with one or two alkyl groups selected from methyl, and ethyl
a ring residue selected from oxacyclohexyl, oxacyclohexenyl,
oxacyclopentyl, oxacyclopentenyl wherein the ring is substituted with
a ketogroup, and wherein the ring may optionally be further substituted
with one or two alkyl groups selected from methyl, and ethyl
oxacyclopentan, substituted oxacyclopentyl
furanyl
thiacyclopentyl, substituted thiacyclopentyl
thiophenyl
azacyclopentyl, substituted azacyclopentyl
pyrrolyl, dihydropyrrolyl
azacyclopentyl, substituted diazacyclopentyl
azathiacyclopentenyl including a dihydrothiazolyl (for example:
2-thiadihydrothiazolyl)
thiazolyl, 2-thiazolyl
alkylthiazolyl including methylthiazolyl, ethylthiazolyl, and
dimethylthiazolyl
azaoxacyclopentyl
oxazolyl, 2-oxazolyl, 5-oxazolyl
oxacyclohexyl
thiacyclohexyl
azacyclohexyl
pyridinyl, alkylpyridinyl, methylpyridinyl, dimethylpyridinyl,
ethylpyridinyl, trimethylpyridinyl, tetramethylpyridinyl,
ethylmethylpyridinyl, ethyldimethylpyridinyl
diazacyclohexyl
pyrazinyl, alkylpyrazinyl, methylpyrazinyl, dimethylpyrazinyl,
trimethylpyrazinyl, ethylmethylpyrazinyl, ethyldimethylpyrazinyl,
ethylpyrazinyl, diethylpyrazinyl, diethylmethylpyrazinyl
azathiacyclohexyl
azaoxacyclohexyl

Group of Compounds with R1, R2 and R3 as Defined Below:

Compounds of formula I with R1 selected from heptyl, nonyl, undecyl, tridecyl, pentadecyl, and heptadecyl, R2=methyl, and R3 selected from a 1,1-hydroxyethyl, 1-oxoethyl, and 1-oxopropyl.

Compounds of formula I with R1 selected from straight-chain C7 to C17 alkyl, straight-chain C7 to C17 alkenyl, branched C7 to C17 alkyl, branched C7 to C17 alkenyl, R2=methyl, and R3 selected from straight-chain C1 to C15 alkyl, straight-chain C1 to C15 alkenyl, branched C1 to C15 alkyl, and branched C1 to C15 alkenyl.

Groups of Compounds with a R2-C-R3 Ring Residue:

The following compounds are compounds wherein the R2-C-R3 part of formula I forms a ring residue which is bound to the two ring oxygen atoms of formula I. For all residues, substituents and double bonds are counted starting from the C atom in position 1 (C¹, compare structural formula below) which is the C atom between R2 and R3. Double bonds may be in cis or trans position. To illustrate, the following structural formula shows a compound of formula I wherein R2-C-R3 is 2,4-dimethyl-4,5-dihydro-(2H)-furanone:

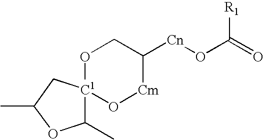

Compounds of formula I with R1 as defined herein-above and R2-C-R3 selected as indicated in the table below.

Compounds of formula I with R1 selected from C7 to C17 alkyl, C7 to C17 alkenyl, C7 to C17 monoalkenyl, C7 to C17 alkadienyl, straight-chain C7 to C17 alkyl, straight-chain C7 to C17 alkenyl, straight-chain C7 to C17 monoalkenyl, straight-chain C7 to C17 alkadienyl, branched C7 to C17 alkyl, branched C7 to C17 alkenyl, branched C7 to C17 monoalkenyl, branched C7 to C17 alkadienyl, and R2-C-R3 selected as indicated in the table below.

Compounds of formula I with R1 selected from C7 to C17 alkyl, C7 to C17 alkenyl, C7 to C17 monoalkenyl, C7 to C17 alkadienyl, and R2-C-R3 selected as indicated in the table below.

Compounds of formula I with R1 selected from C7, C9, C11, C13, C15, or C17 straight-chain alkyl and C7, C9, C11, C13, C15, or C17 branched alkyl

Compounds of formula I with R1 selected from heptyl, nonyl, undecyl, tridecyl, pentadecyl, and heptadecyl, and R2-C-R3 selected as indicated in the table below.

TABLE 2
Groups of R2—C—R3 residues
R2—C—R3 ring residue is selected from:

oxacyclopentan optionally substituted with a residue selected from methyl,
dimethyl, ethyl, hydroxy, and methoxy
oxacyclopentene optionally substituted with a residue selected from
methyl, dimethyl, ethyl, hydroxy, and methoxy
hydroxycyclopentene, hydroxyalkylcyclopentene,
hydroxymethylcyclopentene, hydroxydimethylcyclopentene,
ethylhydroxycyclopentene, and ethylhydroxymethylcyclopentene
thiacyclopentane, alkylthiacyclopentane, and alkyl-3-thia-cyclopentane
oxa-cyclopentene, 2-oxacyclopentene, 3-oxacyclopentene,
alkyloxacylcopentene, alkyl-3-oxacyclopentene,
methyl-3-oxa-cyclopentene, alkyl-3-oxacyclopentene,
dimethyl-3-oxacyclopentene, ethyl-3-oxacyclopentene,
ethylmethyl-3-oxacyclopentene, hydroxyalkyl-3-oxacyclopentene,
hydroxymethyl-3-oxacyclopentene, hydroxydimethyl-3-oxacyclopentene,
hydroxyethyl-3-oxacyclopentene, ethylhydroxymethyl-3-oxacyclopentene
oxacyclopentane, 3-oxacyclopentan, alkyl-3-oxacyclopentan,
methyl-3-oxa-cyclopentan, dimethyl-3-oxacyclopentan,
ethyl-3-oxacyclopentan, ethylmethyl alkyl-3-oxacyclopentan.
alkylcyclopenten, alkenylcyclopenten, alkylalkenylcyclopenten,
methylcylopenten, dimethylcyclopenten, ethylmethylcyclopenten,
propylmethylcyclopenten, butylmethylcyclopenten,
butenylmethylcyclopenten, pentylmethylcyclopenten,
pentenylmethylcyclopenten
alkylcarboxy-alkenyl-cyclopentan, alkylcarboxy-alkyl-cyclopentan,
methylcarboxy-alkenyl-cyclopentan, methylcarboxy-alkyl-cyclopentan,
alkylcarboxy-pentenyl-cyclopentan, alkylcarboxy-pentyl-cyclopentan
methylcarboxy-alkenyl-cyclopentan, and
methylcarboxy-alkyl-cyclopentan, and wherein the alkyl is selected
from methyl, ethyl, propyl, butyl, pentyl, pentenyl, hexyl, heptyl,
and octyl
oxo-alkylcyclohexen, oxo-methylcyclohexen, oxo-trimethylcyclohexen,
oxo-ethylcyclohexen, oxo-ethylmethylcyclohexen, oxo-propylcyclohexen,
oxo-propylmethylcyclohexen, oxo-isopropylcyclohexen,
oxo-isopropylmethylcyclohexen, oxo-thiopropylcyclohexen,
oxo-thiopropylmethylcyclohexen
alkylcyclohexen, methylcyclohexen, trimethylcyclohexen,
ethylcyclohexen, ethylmethylcyclohexen, propylcyclohexen,
propylmethylcyclohexen, isopropylcyclohexen,
isopropylmethylcyclohexen, thiopropylcyclohexen,
thiopropylmethylcyclohexen,
alkylcyclohexan, methylcyclohexan, trimethylcyclohexan,
ethylcyclohexan, ethylmethylcyclohexan, propylcyclohexan,
propylmethylcyclohexan, isopropylcyclohexan,
isopropylmethylcyclohexan, thiopropylcyclohexan,
thiopropylmethylcyclohexan

In the Following, Further Groups of Compounds of Formula I Comprising R2-C-R3 Useful in the Invention are Given:

A compound according to formula I wherein the R2-C-R3 substituted cyclopentene residue is selected from the group consisting of 2-hydroxy-3-methylcyclopent-2-ene, 2-hydroxy-4-methylcyclopent-2-ene, 3-ethyl-2-hydroxycyclopent-2-ene, 2-hydroxy-3,4-dimethylcyclopent-2-ene, 3-ethyl-2-hydroxycyclopent-2-ene, 4-ethyl-2-hydroxy-3-methylcyclopent-2-ene. These compounds will release hydroxy-alkyl-cyclopentenones.

A compound according to formula I wherein the R2-C-R3 thiacycloalkane ring residue is 3-thiacyclopentane.

A compound according to formula I wherein the R2-C-R3 substituted oxacyclopentene ring residue is selected from 2-methoxy-4-oxa-3,5-dimethylcyclopent-2-ene, 2-hydroxy-3,5-dimethyl-4-oxacyclopent-2-ene, 5-ethyl-2-hydroxy-3-methyl-4-oxa-pent-2-ene, 2,5-dimethyl-3-oxa-cyclopent-4-ene.

A compound according to formula I wherein the R2-C-R3 substituted oxacyclopentane ring residue is selected from the group consisting of 2,4-dimethyl-3-oxa-cyclopentan, and 2-methyl-3-oxa-cyclopentan.

A compound according to formula I wherein the R2-C-R3 alkyl/alkenyl/alkylalkenyl substituted cyclopentene ring residue is selected from 2-((Z)-pent-2-enyl)-3-methylcyclopent-2-en, 2-(-pent-2-enyl)-3-methylcyclopent-2-en.

A compound according to formula I wherein the R2-C-R3 alkylcarboxyalky/alkylcarboxyalkenyl-cylopentan ring residue is selected from 3-(methoxycarbonylmethyl)-2-pentyl-cyclopentan, 3-(methoxycarbonylmethyl)-2(Z)-pent-2-enyl-cyclopentan, cis 3-(methoxycarbonylmethyl)-2(Z)-pent-2-enyl-cyclopentan, trans 3-(methoxycarbonylmethyl)-2(Z)-pent-2-enyl-cyclopentan, and 2R3S-cis-3-(methoxycarbonylmethyl)-2(Z)-pentyl-cyclopentan.

A compound according to formula I wherein the R2-C-R3 alkyl/thioalkyl substituted oxo-cyclohexan/hexene ring residue is selected from 4-oxo-3,5,5-trimethylcyclohex-2-en, 3,5,5-trimethylcyclohex-2-en, 4-isopropyl-cyclohex-2-en, 2-isopropyl-5-methylcyclohexan, 4-isopropylcyclohexan, 2-(2-thiopropan-2-yl)-5-methylcyclohexan.

A compound according to formula I wherein R2 is H and R3 is a methyl-cyclohexdienyl including but not limited to 2,6,6-trimethyl-cyclohex-1,3-dienyl. Alternatively, R2 may be selected from methyl, ethyl, propyl, butyl, butenyl, pentyl, hexyl, heptyl.

The Following Groups of Compounds are Example Groups of Precursor Compounds that Will Release Aldehyde Flavors:

A compound according to formula I wherein R2 is H and R3 is selected from phenyl, alkylphenyl, and substituted phenyl with the substituent selected from OH, and alkoxy.

A compound according to formula I wherein R2 is H and wherein R3 is an alkylphenyl selected from methylphenyl, ethylphenyl, propylphenyl, isopropylphenyl, butylphenyl, isobutylphenyl, and pentylphenyl.

A compound according to formula I wherein R2 is H and wherein R3 is a substituted phenyl selected from hydroxyphenyl, methoxyphenyl, and ethoxyphenyl.

A compound according to formula I wherein R2 is H and wherein R3 is a doubly substituted phenyl selected from hydroxy-alkoxyphenyl, hydroxymethoxyphenyl, alkoxy-alkylphenyl, methoxy-alkylphenyl, hydroxy-alkylphenyl, and hydroxymethoxyphenyl.

A compound according to formula I wherein R2 is H and wherein R2-C-R3 is selected from phenyl, 4-methylphenyl, 4-methoxy phenyl, 4-hydroxyphenyl, 4-isopropylphenyl, 4-hydroxy-3-methoxyphenyl, and piperonyl.

A compound according to formula I wherein R2 is H and wherein R3 is selected from furanyl, alkylfuranyl, methylfuranyl, ethylfuranly, propylfuranyl, isopropylfuranyl, butylfuranyl, isobutylfuranyl.

A compound according to formula I wherein R2 is H and wherein R3 is selected from furan-2-yl, 5-methylfuran-2-yl, 3-methylfuran-2-yl, furan-3-yl (from: acetyl NSO).

A compound according to formula I wherein R2 is H and wherein R3 is selected from thiophenyl, alkylthiophenyl, methylthiophenyl, ethylthiophenyl, dialkylthiophenyl, dimethylthiophenyl, ethylmethylthiophenyl.

A compound according to formula I wherein R2 is H and wherein R3 is selected from thiophen-2-yl, 5-methylthiophen-2-yl, 5-ethylthiophen-2-yl, thiophen-3-yl, 2-methylthiophen-3-yl, 2,5-dimethylthiophen-3yl.

A compound according to formula I wherein R2 is H and wherein R3 is selected from phenylalkyl and phenylalkenyl.

A compound according to formula I wherein R2 is H and wherein R3 is a phenylalkyl selected from benzyl, phenylethyl, phenylpropyl, phenylbutyl, phenylpentyl, phenylhexyl, phenylheptyl, phenyloctyl, phenylnonyl.

A compound according to formula I wherein R2 is H and wherein R3 is a phenylalkenyl selected from phenylethenyl, phenylpropenyl, phenylbutenyl, phenylpentenyl, phenylhexenyl, phenylheptenyl, phenyloctenyl, phenylnonenyl.

A compound according to formula I wherein R2 is H and wherein R3 is selected from 1-phenylethyl, 4-methyl-2-phenylbut-2-enyl, (Z)-4-methyl-2-phenylbut-2-enyl, 5-methyl-2-phenylpent-2-enyl, (Z)-5-methyl-2-phenylpent-2-enyl, benzyl, 2-phenylprop-2-enyl, (Z)-2-phenylprop-2-enyl, 1-phenylvinyl, 2-phenylvinyl.

A compound according to formula I wherein R2 is H and wherein R3 is selected from alkylthioalkyl, alkylthioalkenyl, alkyldisulfanylalkyl, alkyldisulfanylalkenyl, methylthioalkyl, methyl-methylthioalkyl, methylthioalkenyl, methyl-methylthioalkenyl, methylthioalkyl, methyl-methylthioalkyl, methylthioalkenyl, methyl-methylthioalkenyl, methyidisulfanylalkyl, methyl-methyldisulfanylalkyl, methyidisulfanylalkenyl, methyl-methyldisulfanylalkenyl.

A compound according to formula I wherein R2 is H and wherein R3 is selected from 3-(methylthio)propyl, 2-(methylthio)propyl, 4-(methylthio)but-2-en-2-yl, (Z)-4-(methylthio)but-2-en-2-yl, 2-methyl-5-(methylthio)pent-2-en-2-yl, (E)-2-methyl-5-(methylthio)pent-2-en-2-yl, 1-((methyl(thio)methyl)-but-2-en-2-yl, (Z)-1-((methyl(thio)methyl)-but-2-en-2-yl, 2-(methylthio)ethyl, and 2-(2-methyl(disulfanyl)-prop-2-yl.

A compound according to formula I wherein R2 is H and wherein R3 has a maximum number of C atoms of 12 and is selected from singly branched hydroxyalkyl, doubly branched hydroxyalkyl, multiply branched hydroxyalkyl, hydroxydialkylalkyl, hydroxydimethylalkyl, hydroxydimethylbutyl, hydroxydimethylpentyl, hydroxydimethylhexyl, hydroxydimethylheptyl, hydroxydimethyloctyl, hydroxydimethyinonyl.

A compound according to formula I wherein R2 is H and wherein R3 is selected from 6-hydroxy-3,6-dimethylheptyl.

Alternatively, the groups of compounds above may have instead of the indicated R2 (H) a R2 selected from methyl, ethyl, propyl, butyl, pentyl, hexyl, heptyl, octyl, nonyl, decyl. These groups of precursor compounds will release, instead of the flavor aldehydes, flavor ketone compounds.

For the groups of precursor compounds releasing flavor aldehydes described above, or the alternative groups of compounds described above that release flavor ketones, R1 is selected as indicated herein-above or selected from heptyl, nonyl, undecyl, tridecyl, pentadecyl, and heptadecyl.

Groups of Precursor Compounds that Release Acetyl Flavor Compounds:

The following groups of compounds may be formed by reacting flavor compounds with a ketone to form the R2-C-R3 part of the compound of formula I, wherein C is the former carbonyl C-atom of the ketone educt. The se precursor compounds will release acetyl flavor compounds accordingly. The resulting R3 residues are indicated below:

A compound according to formula I wherein R2 is methyl and R3 is selected from pyrazinyl, alkylpyrazinyl, methylpyrazinyl, dimethylpyrazinyl, trimethylpyrazinyl, ethylmethylpyrazinyl, ethyidimethylpyrazinyl, ethylpyrazinyl, diethylpyrazinyl, diethylmethylpyrazinyl.

A compound according to formula I wherein R2 is methyl and R3 is selected from pyrazinyl, 3-methylpyrazin-2-yl, 6-methylpyrazin-2-yl, 2,3-dimethylpyrazin-6-yl, 3,5-dimethylpyrazin-2-yl, 5-dimethylpyrazin-2-yl, 2-ethyl-pyrazin-6-yl, 5-ethyl-3-methylpyrazin-2-yl, 2-ethyl-5-methylpyrazin-3-yl, 2-ethyl-5,6-dimethylpyrazin-3-yl, 2-ethyl-3,5-dimethylpyrazin-6-yl, 3-ethyl-5-methylpyrazin-2-yl, 2-ethyl-pyrazin-5-yl, 2-ethyl-pyrazin-3-yl.

A compound according to formula I wherein R2 is methyl and R3 is selected from alkylpyridinyl, methylpyridinyl, dimethylpyridinyl, trimethylpyridinyl, tetramethylpyridinyl, ethylmethylpyridinyl, ethyidimethylpyridinyl.

A compound according to formula I wherein R2 is methyl and R3 is selected from pyridin-2-yl, 3-methylpyridin-2-yl, 5-methylpyridin-2-yl, 6-methylpyridin-2-yl, 3-ethylpyridin-2-yl, 3,5-dimethylpyridin-2-yl, 5,6-dimethylpyridin-2-yl, 3-ethyl-5-methylpyridin-2-yl, 3-ethyl-6-methylpyridin-2-yl, 3,5,6-trimethylpyridin-2-yl, 3-ethyl-5,6,-dimethylpyridin-2-yl, 2-ethyl-3,5-dimethylpyridin-6-yl, pyridin-3-yl, 2-methylpyridin-3-yl, 5-methylpyridin-3-yl, 6-methylpyridin-3-yl, 2,3-dimethylpyridin-5-yl, 2,6-dimethylpyridin-3-yl, 2,3,4,5-tetrahydropyridin-6-yl, 1,2,3,4-tetrahydropyridin-6-yl.

A compound according to formula I wherein R2 is methyl and R3 is selected from oxazolyl, thiazolyl, dihydro-thiazolyl.

A compound according to formula I wherein R2 is methyl and R3 is selected from oxazol-2-yl, oxazol-4-yl, oxazol-5-yl, thiazol-2-yl, thiazol-4-yl, thiazol-5-yl, 4,5-dihydro-thiazol-2-yl, 2,5-dihydro-thiazol-2-yl.

A compound according to formula I wherein R2 is ethyl and R3 is selected from oxazolyl, thiazolyl, dihydro-thiazolyl.

A compound according to formula I wherein R2 is ethyl and R3 is selected from thiazol-2-yl, oxazol-2-yl, oxazol-4-yl, oxazol-5-yl, thiazol-2-yl, thiazol-4-yl, thiazol-5-yl, 4,5-dihydro-thiazol-2-yl, 2,5-dihydro-thiazol-2-yl.

A compound according to formula I wherein R2 is methyl and R3 is selected from pyrrolyl, and dihydropyrrolyl.

A compound according to formula I wherein R2 is methyl and R3 is selected from 1H-pyrrol-2-yl, 1H-pyrrol-3-yl, 3,4-dihydro-2H-pyrrol-5-yl, 2,3-dihydro-1H-pyrrol-5-yl.

A compound according to formula I wherein R2 is methyl and R3 is selected from thioalkyl, thiomethyl, thioethyl, thiopropyl, thiobutyl, thiopentyl, thiomethylethyl, thiomethylpropyl, thiomethylbutyl, alkyl(methylthio)alkyl, methyl(methylthio)alkyl, methyl(methylthio)ethyl, methyl(methylthio)propyl, methyl(methylthio)butyl.

A compound according to formula I wherein R2 is methyl and R3 is selected from l-thiopropyl, 2-thiopropyl, 3-thiopropyl, thiomethyl, 2-thio-2-methylpropyl, 2-methyl-2-(methylthio)propyl.

A compound according to formula I wherein R2 is methyl and R3 is selected from furanyl-substituted straight-chain or branched alkyl including methyl, ethyl, propyl, butyl, and pentyl, optionally substituted with one or more of OH, O, and SH; straight-chain or branched furanyl-substituted alkylthioalkyl, and furanyl-substituted methylthioalkyl

A compound according to formula I wherein R2 is methyl and R3 is selected from 1-((furan-2-yl)methylthio)-1-hydroxyethyl, and 2-((furan-2-yl)methylthio)-2-methylpropyl.

A compound according to formula I wherein R2 is methyl and R3 is selected from straight-chain or branched substituted alkyl substituted with one or more of O, and OH, a straight-chain or branched substituted alkyl substituted with one or more of O and OH wherein the alkyl is selected from ethyl, propyl, butyl, pentyl, hexyl, heptyl, and octyl, a straight-chain or branched (alkoxycarbonyl)alkyl, a straight-chain or branched (alkoxycarbonyl)alkyl wherein the alkyl is selected from ethyl, propyl, butyl, pentyl, hexyl, heptyl, and octyl.

A compound according to formula I wherein R2 is methyl and R3 is selected from 1-oxopentyl, 1-oxobutyl, 1-oxopropyl, 3-oxo-oct-2-yl, 2-(ethoxycarbonyl)ethyl.

A compound according to formula I wherein R2 is methyl and R3 is selected from alkylphenyl, phenylalkyl, C9 arylalkyl, C10 arylalkyl, phenylalkenyl, C9 arylalkenyl, C10 arylalkenyl, methoxyphenyl, methoxyphenylalkyl, methoxyphenylalkenyl, hydroxyphenylalkyl, hydroxyphenylalkenyl, hydroxyphenylalkyl.

A compound according to formula I wherein R2 is methyl and R3 is selected from 4-methylphenyl, 2-phenylethenyl, 4-methoxyphenyl, 2-(4-methoxyphenyl)ethyl, 2-(4-hydroxyphenyl)ethyl, and benzoyl.

Alternatively, the groups of compounds above may have instead of the indicated R2 (methyl) a R2 selected from H (in which case the reacting flavor compound/educt that forms the R2-C-R3 residue of the precursor and the released flavor compound is an aldehyde), ethyl, propyl, butyl, pentyl, hexyl, heptyl, octyl, pentyl, decyl.

For the groups of precursor compounds that release flavor acetyls described above, R1 is selected as indicated herein-above or selected from heptyl, nonyl, undecyl, tridecyl, pentadecyl, and heptadecyl.

The Following Groups of Compounds Will Release Alkylaldehyde and Alkenylaldehyde Flavors:

A compound according to formula I wherein R2 is H and R3 is selected from methyl, ethyl, propyl, butyl, pentyl, hexyl, heptyl, octyl, nonyl, decyl, undecyl, dodecyl, tridecyl.

A compound according to formula I wherein R2 is H and R3 is a singly branched alkyl selected from isopropyl, methylbutyl, methylpentyl, methylhexyl, methylheptyl, methyloctyl, methylnonyl, methyldecyl, methylundecyl, methyldodecyl, methyltridecyl.

A compound according to formula I wherein R2 is H and R3 is 11-methyl-dodecyl.

A compound according to formula I wherein R2 is H and R3 is 1-methyl-propyl.

A compound according to formula I wherein R2 is H and R3 is 2-methyl-propyl.

A compound according to formula I wherein R2 is H and R3 is an alkenyl with a single double bond selected from propenyl, butenyl, pentenyl, hexenyl, heptenyl, octenyl, nonenyl, decenyl, undecenyl, dodecenyl, tridecenyl.

A compound according to formula I wherein R2 is H and R3 is an alkenyl with a single double bond selected from prop-1-enyl, but-1-enyl, buten-2-yl, but-3-enyl, pent-1-enyl, pent-2-enyl, pent-3-enyl, hex-1-enyl, hex-3-enyl, hept-1-enyl, oct-1-enyl, oct-5-enyl, non-1-enyl, non-3-enyl, non-8-enyl, dec-1-enyl, dec-8-enyl, dec-9-enyl, undec-1-enyl, dodec-1-enyl, tridec-1-enyl.

A compound according to formula I wherein R2 is H and R3 is an alkenyl with two double bonds selected from penta-1,3-dienyl, hexa-1,3-dienyl, hepta-1,3-dienyl, octa-1,3-dienyl, octa-1,5-dienyl, nona-1,3-dienyl, deca-1,3-dienyl, undeca-1,3-dienyl, dodeca-1,3-dienyl, trideca-1,3-dienyl, octa-1,5-dienyl.

A compound according to formula I wherein R2 is H and R3 is an alkenyl selected from an alkenyl with multiple double bonds, and an alkenyl with three double bonds.

A compound according to formula I wherein R2 is H and R3 is an alkenyl with three double bonds octatrienyl, nonatrienyl, decatrienyl, undecatrienyl, dodecatrienyl.

A compound according to formula I wherein R2 is H and R3 is an alkenyl with three double bonds selected from octa-1,3,5-trienyl, nona-1,3,5-trienyl, deca-1,3,5-trienyl, undeca-1,3,5-trienyl, dodeca1,3,5-trienyl.

A compound according to formula I wherein R2 is H and R3 is an alkyl or alkenyl substituted with one, two, or more methyl groups.

A compound according to formula I wherein R2 is H and R3 is an alkyl with a methyl group including but not limited to 1-methyl-methyl, 1-methyl-ethyl, 1-methyl-propyl, 1-methyl-butyl, 1-methyl-pentyl, 1-methyl-hexyl, 1-methyl-heptyl, 1-methyl-octyl, 1-methyl-nonyl.

A compound according to formula I wherein R2 is H and R3 is an alkenyl with a methyl group including but not limited to 2-methyl-prop-2-enyl, 3-methylbut-1-enyl.

A compound according to formula I wherein R2 is H and R3 is an alkyl with two methyl groups including but not limited to 3,6-dimethylheptyl.

A compound according to formula I wherein R2 is H and R3 is an alkenyl with two double bonds and two methyl groups including but not limited to 3,6-dimethylhepta-1,5-dienyl.

A compound according to formula I wherein R2 is H and R3 is an alkenyl with a double bond and two methyl groups including but not limited to 3,6-dimethylhept-5-enyl.

A compound according to formula I wherein R2 is H and R3 is a branched alkyl or alkenyl substituted with one or two ethyl groups.

A compound according to formula I wherein R2 is H and R3 is an alkyl substituted with one ethyl group including but not limited to 1-ethyl-propyl.

A compound according to formula I wherein R2 is H and R3 is an alk-3-yl or alken-3-yl substituted with one or more methyl groups, including but not limited to 2,6-dimethyl.hept-3-en-3-yl.

The above-described alkyl and alkenyl R3 residues include but are not limited to alk-1-yl, alk-1-enyl, alk-2-yl, alk-2-enyl, alk-3-yl, alk-3-enyl, alk-4-yl, and alk-4-enyl residues.

A compound according to formula I wherein R2 is H and R3 is pent-2-en-2-yl.

Alternatively to the above-described groups of compounds wherein R3=alkyl or alkenyl, instead of the indicated R2 (H), R2 may be selected from methyl, ethyl, propyl, butyl, pentyl, hexyl, heptyl, octyl, pentyl, decyl, undecyl, dodecyl, tridecyl, tetradecyl, pentadecyl; still alternatively, R2 may be selected from H, methyl, ethyl, butyl, 2-butenyl, or vinyl. For the above-described compounds, R1 is selected as indicated herein-above or selected from heptyl, nonyl, undecyl, tridecyl, pentadecyl, and heptadecyl.

For the above-described groups of compounds wherein R3 is alkenyl, double bonds may be in cis or trans position.

The Following Groups of Precursor Compounds Will Release Alkylketone or Alkenylketone Flavors:

A compound according to formula I wherein R2 is methyl and R3 is an alkyl selected from ethyl, propyl, butyl, pentyl, hexyl, heptyl, octyl, nonyl, decyl, undecyl, dodecyl, tridecyl.

A compound according to formula I wherein R2 is methyl and R3 is an alkenyl selected from ethenyl, propenyl, butenyl, pentenyl, hexenyl, heptenyl, octenyl, nonenyl, decenyl, undecenyl, dodecenyl, tridecenyl, tetradecenyl, pentadecenyl.

A compound according to formula I wherein R2 is methyl and R3 is an alkenyl selected from prop-1-enyl, but-1-enyl, pent-1-enyl, hex-1-enyl, hept-1-enyl.

A compound according to formula I wherein R2 is methyl and R3 is a methylalkenyl selected from methylethenyl, methylpropenyl, methylbutenyl, methylpentenyl, methylhexenyl, methylheptenyl, methyloctenyl, methylnonenyl, methyldecenyl, methylundecenyl, methyldodecenyl, methyltridecenyl.

A compound according to formula I wherein R2 is methyl and R3 is a methylalkenyl as defined above including but not limited to 4-methyl-pent-3-enyl, 4-methyl-penta-1,3-dienyl.

A compound according to formula I wherein R2 is methyl and R3 is an alkadienyl selected from butadienyl, pentadienyl, hexadienyl, heptadienyl, octadienyl, nonadienyl, decadienyl, undecadienyl, dodecadienyl, tridecadienyl, tetradecadienyl, pentadecadienyl.

A compound according to formula I wherein R2 is methyl and R3 is an alkadienyl selected from penta-1,3-dienyl, hexa-1,3-dienyl, hepta-1,3-dienyl, octa-1,3-dienyl.

A compound according to formula I wherein R2 is methyl and R3 is a hydroxy-alkyl including but not limited to hydroxy-ethyl, hydroxy-propyl, hydroxy-butyl, hydroxy-pentyl, hydroxy-hexyl, hydroxy-heptyl, 1-hydroxy-ethyl, 1-hydroxy-propyl, 1-hydroxy-butyl, 1-hydroxy-pentyl, 1-hydroxy-hexyl, 1-hydroxyheptyl.

A compound according to formula I wherein R2 is methyl and R3 is an oxo-alkyl including but not limited to oxo-ethyl, oxo-propyl, oxo-butyl, oxo-pentyl, oxo-hexyl, oxo-heptyl, 1-oxo-ethyl, 1-oxo-propyl, 1-oxo-butyl, 1-oxo-pentyl, 1-oxo-hexyl, 1-oxoheptyl.

Alternatively to the above-described groups of compounds wherein R3 is optionally substituted alkyl or alkenyl, instead of the indicated R2 (methyl), R2 may be selected from methyl, ethyl, propyl, butyl, pentyl, hexyl, heptyl, octyl, pentyl, decyl, undecyl, dodecyl, tridecyl, tetradecyl, pentadecyl; still alternatively, R2 may be selected from H, methyl, ethyl, butyl, 2-butenyl, or vinyl.

A compound according to formula I wherein R2 is ethyl and R3 is selected from ethyl, propyl, prop-1-enyl, butyl, pentyl, hexyl, heptyl.

A compound according to formula I wherein R2 is 2-butyl and R3 is prop-1-enyl

A compound according to formula I wherein R2 is vinyl and R3 is selected from ethyl, propyl, prop-1-enyl, butyl, pentyl, hexyl, heptyl.

For the above-described groups of compounds wherein R3 is optionally substituted alkyl or alkenyl, R1 is selected as indicated herein-above or selected from heptyl, nonyl, undecyl, tridecyl, pentadecyl, and heptadecyl.

For the above-described groups of compounds wherein R3 is optionally substituted alkenyl, double bonds may be in cis or trans position.

Some examples of useful flavor precursor compounds are given in table 2 below, further example compounds can be found in the examples herein-below:

| | | |
|---|---|---|
| 1 | 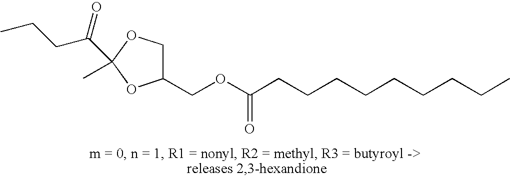 | example 3 |
| 2 | 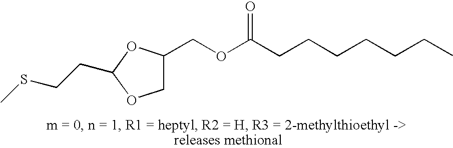 | example 4 |
| 3 | 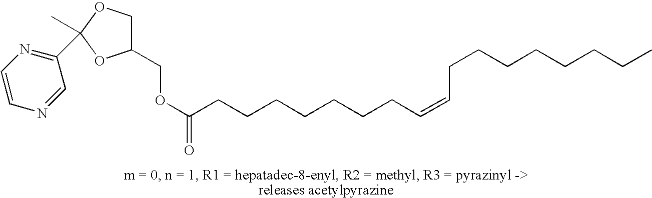 | example 5 |
| 4 | 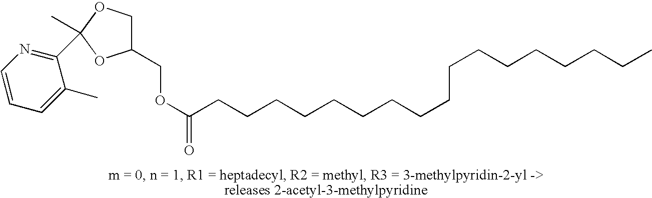 | example 6 |
| 5 | 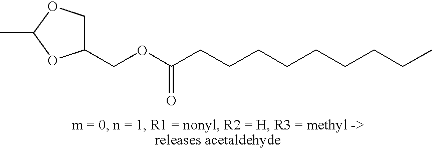 | example 7 |
| 6 | 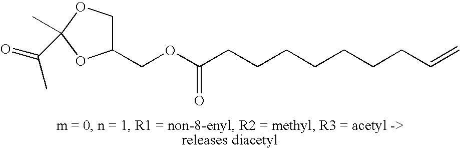 | example 8 |
| 7 | 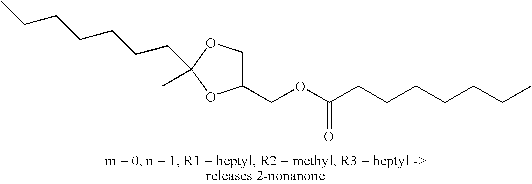 | example 9 |
| 8 | 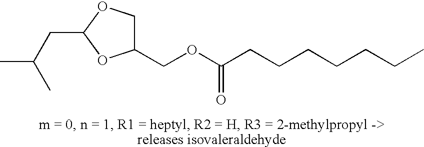 | example 10 |
| 9 | 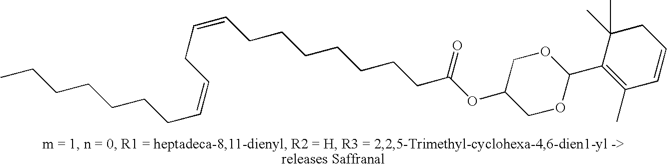 | |
| 10 | 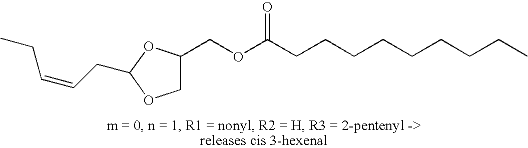 | |
| 11 | 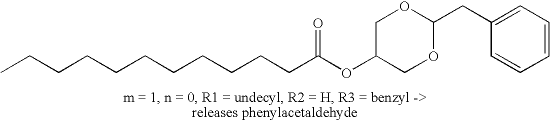 | |
| 12 | 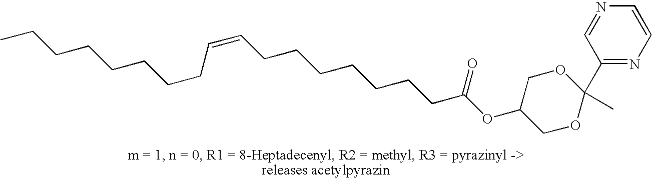 | |
| 13 | 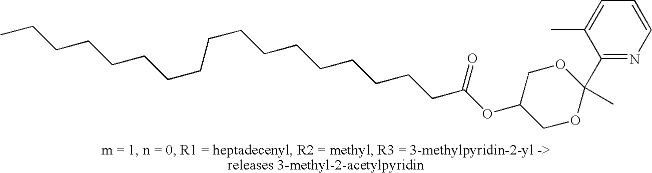 | |
| 14 | 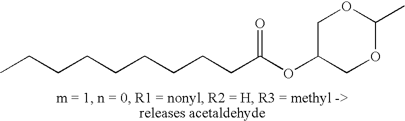 | |
| 15 | 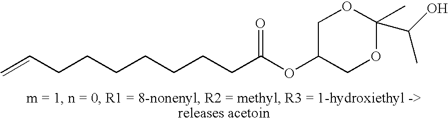 | |
| 16 | 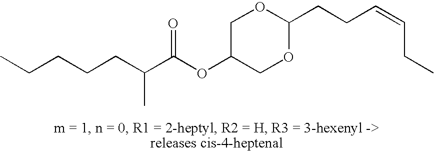 | |
| 17 | 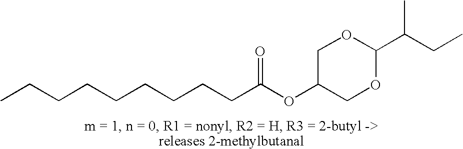 | |
| 18 | 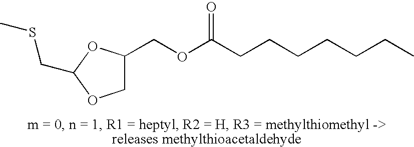 | |
| 19 | 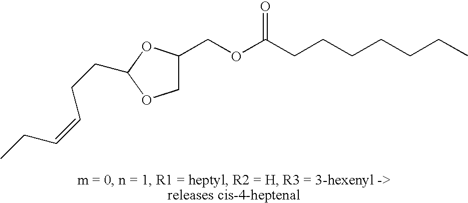 | |
| 20 | 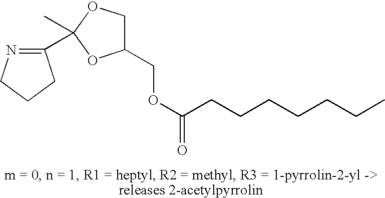 | |
| 21 | 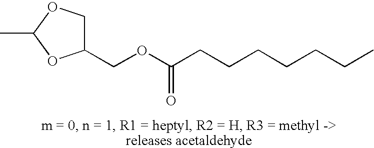 | |
| 22 | 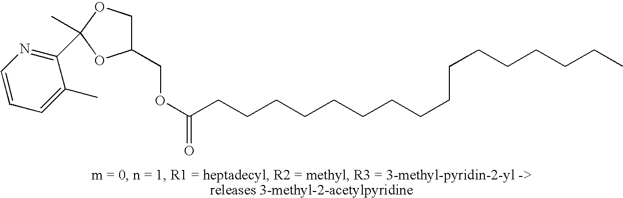 | |
| 23 | 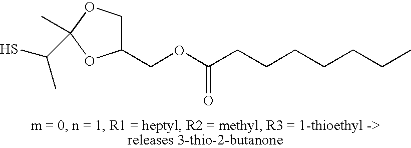 | |
| 24 | 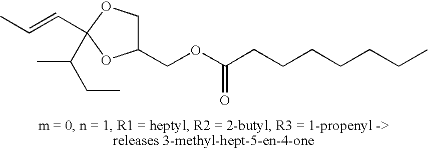 | |
| 25 | 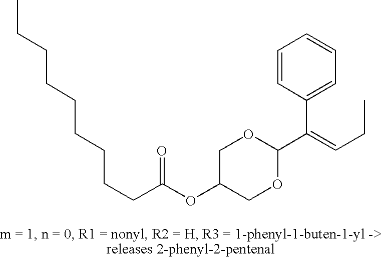 | |
| 26 | 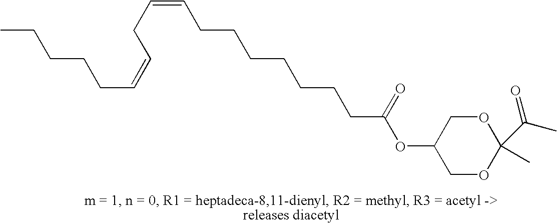 | |
| 27 | 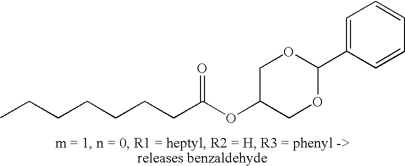 | |
| 28 | 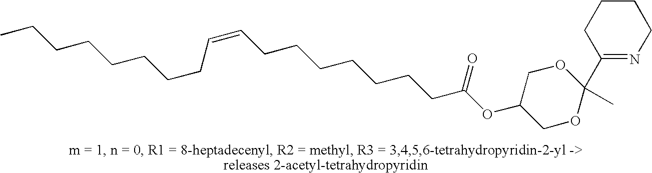 | |
| 29 | 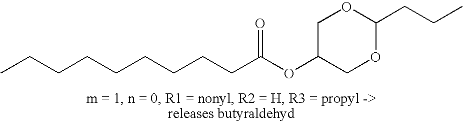 | |
| 30 | 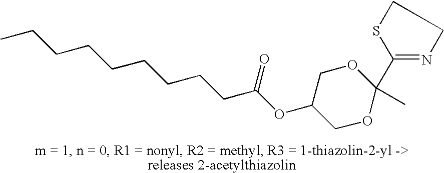 | |
| 31 | 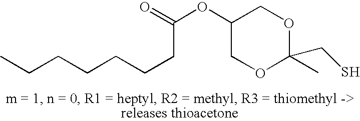 | |
| 32 | 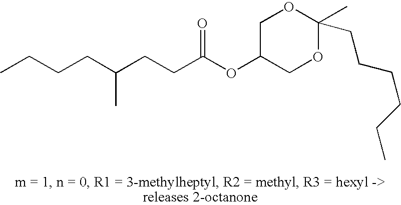 | |
| 33 | 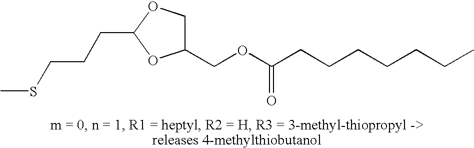 | |
| 34 | 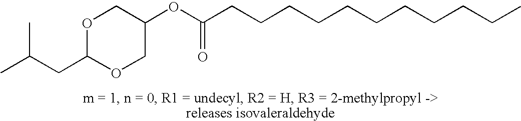 | |
| 35 | 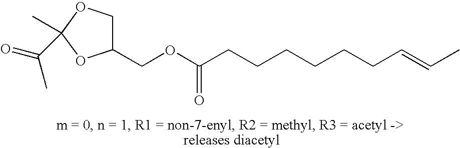 | |

Alkyl residues of formula I include, without limitation, methyl (C1), ethyl (C2), propyl and/or isopropyl (C3), butyl and/or isobutyl (C4), pentyl and/or isopentyl (C5), hexyl and/or isohexyl (C6), heptyl and/or isoheptyl (C7), octyl and/or isooctyl (C8), nonyl and/or isobornyl (C9), decyl and/or isodecyl (C10), undecyl and/or isodecyl (C11), dodecyl and/or isododecyl (C12), tridecyl and/or isotridecyl (C13), tetradecyl and/or isotetradecyl (C14), pentadecyl and/or isopentadecyl (C15), hexadecyl and/or isohexadecyl (C16), heptadecyl and/or isoheptadecyl (C17), octadecyl and/or isooctadecyl (C18).

Alkenyl residues include, without limitation, ethenyl (C2), propenyl and/or isopropenyl (C3), butenyl and/or isobutenyl (C4), pentenyl and/or isopentenyl (C5), hexenyl and/or isohexenyl (C6), heptenyl and/or isoheptenyl (C7), octenyl and/or isooctenyl (C8), nonenyl and/or isononenyl (C9), decenyl and/or isodecenyl (C10), undecenyl and/or isoundecenyl (C11), dodecenyl and/or isododecenyl (C12), tridecenyl and/or isotridecenyl (C13), tetradecenyl and/or isotetradecenyl (C14), pentadecenyl and/or isopentadecenyl (C15).

The flavor precursor can be added directly to a food product, or can be provided as a flavor composition to be added to food products.

The term “food product” as used herein includes any food product, for example, without limitation, cereal products, rice products, tapioca products, sago products, baker's products, biscuit products, pastry products, bread products, confectionery products, desert products, gums, chewing gums, chocolates, ices, honey products, treacle products, yeast products, baking-powder, salt and spice products, savory products, mustard products, vinegar products, sauces (condiments), tobacco products, cigars, cigarettes, processed foods, cooked fruits and vegetable products, meat and meat products, jellies, jams, fruit sauces, egg products, milk and dairy products, cheese products, butter and butter substitute products, milk substitute products, soy products, edible oils and fat products, medicaments, beverages, alcoholic drinks, beers, soft drinks, mineral and aerated waters and other non-alcoholic drinks, fruit drinks, fruit juices, coffee, artificial coffee, tea, cocoa, including forms requiring reconstitution, food extracts, plant extracts, meat extracts, condiments, sweeteners, nutraceuticals, gelatins, pharmaceutical and non-pharmaceutical gums, tablets, lozenges, drops, emulsions, elixirs, syrups and other preparations for making beverages.

The flavor composition may comprise well known food additives, for example, without limitation, solvents, binders, diluents, disintegranting agents, lubricants, flavoring agents, coloring agents, preservatives, antioxidants, emulsifiers, stabilisers, flavor-enhancers, sweetening agents, anti-caking agents, and the like. Examples of such carriers or diluents for flavor or fragrance compounds may be found e.g. in Perfume and Flavor Materials of Natural Origin, S. Arctander, Ed., Elizabeth, N.J., 1960; in “Perfume and Flavor Chemicals”, S. Arctander, Ed., Vol. I & II, Allured Publishing Corporation, Carol Stream, USA, 1994; in “Flavourings”, E. Ziegler and H. Ziegler (ed.), Wiley-VCH Weinheim, 1998, and “CTFA Cosmetic Ingredient Handbook”, J. M. Nikitakis (ed.), 1st ed., The Cosmetic, Toiletry and Fragrance Association, Inc., Washington, 1988.

The flavor composition may be added in any suitable form, for example as a liquid, as a paste, or in encapsulated form bound to or coated onto carriers/particles or as a powder.

There now follows a series of non-limiting examples that serve to illustrate the invention.

### Examples

Unless stated otherwise, all % indicated are in wt/wt.

### Example 1

### Preparation of Precursor Compounds from Glycerin-Monodecanoate and Release of Aroma Compound by Heat Hydrolysis or Enzyme/Acid Catalysis in the Mouth

The precursor is prepared as follows. 1 g of Glycerin-monodecanoate (also known as CAS 2277-23-8 or 1-Monocaprin, commercially available from Indofine) and a suitable amount (500 mg unless otherwise stated) of an aroma compound are dissolved in 20 ml hexane with a trace of HCl conc. as catalyst in a Dean-Stark trap and boiled for about 2 hours until the formation of water stops. To the mixture, 0.5 ml of a saturated brine solution is added, and the mixture is shaken. The organic phase (hexane) is separated and dried with a small amount of Magnesium sulfate. The hexane is distilled off under vacuum. The resulting precursor material is further purified by vacuum distillation, and may be further purified by chromatography.

When the precursor is boiled in water, a strong aroma (the aroma of the reacted aroma compound that is being hydrolysed) is released.

Upon tasting of the precursor (1-10 ppm) in an aqueous 5% sugar solution or a 0.3% salt solution at room temperature, a weak, bland aroma that slowly develops the aroma corresponding to the employed aroma compound is perceived.

### Example 2

### (2-((Z)-pent-2-enyl)-1,3-dioxolan-4-yl)methyl decanoate

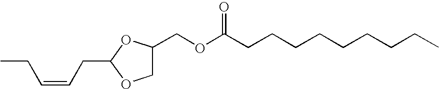

The precursor is a compound of formula I with n=1, m=0, R1=nonyl, R2=H, R3=2-pentenyl and is able to release cis 3-hexenal.

The precursor is prepared using Glycerin-monodecanoate and cis 3-hexenal as described in example 1. The purified precursor is a paste with a weak, green odor.

When the precursor is boiled in water, a strong, fresh, green, gras- or apple-like aroma (the aroma of cis 3-hexenal) is released.

Upon tasting of the precursor (1 ppm) in an aqueous 5% sugar solution at room temperature, a weak, bland aroma that slowly develops a fresh green aroma is perceived.

### Example 3

### (2-butyryl-2-methyl-1,3-dioxolan-4-yl)methyl decanoate

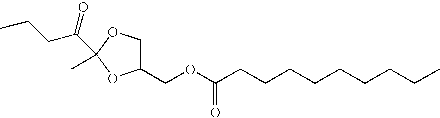

The precursor is a compound of formula I with n=1, m=0, R1=nonyl, R2=methyl, R3=butyroyl and is able to release 2,3-hexandione.

The precursor is prepared using Glycerin-monodecanoate and 2,3-hexandione as described in example 1. The purified precursor is a clear, yellow paste with a weak, buttery odor.

When the precursor is boiled in water, a fresh, buttery, creamy, fruity aroma (the aroma of 2,3-hexandione) is released.

Upon tasting of the precursor (1 ppm) in an aqueous 5% sugar solution at room temperature, a weak, bland aroma that slowly develops a creamy, fruity aroma is perceived.

### Example 4

### (2-(2-(methylthio)ethyl)-1,3-dioxolan-4-yl)methyl octanoate

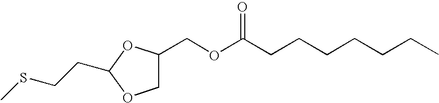

The precursor compound is a compound of formula I with n=1,m=0, R1=heptyl, R2=H, R3=2-methylthioethyl and is able to release methional.

The precursor is prepared using Glycerin-monooctanoate and methional as described in example 1. The purified precursor is a clear, yellowish paste with a sulfurous odor.

When the precursor is boiled in water, a strong sulfuruos, potato, cooked tomato aroma (the aroma of methional) is released.

Upon tasting of the precursor in an aqueous 0.3% salt solution at room temperature, a weak, bland aroma that slowly develops a fresh cooked potato and tomato impression is perceived.

### Example 5

### (2-methyl-2-(pyrazin-2-yl)-1,3-dioxolan-4-yl)methyl oleate

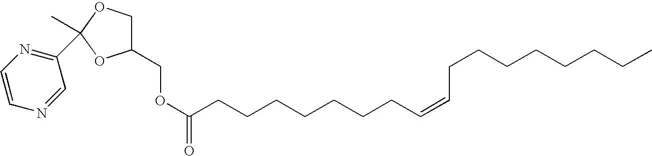

The precursor compound is a compound of formula I with n=1, m=0, R1=heptadec-8-enyl, R2=methyl, R3=pyrazinyl and is able to release acetylpyrazine.

The precursor is prepared using Glycerin-monooleate and acetylpyrazine as described in example 1. The purified precursor is a creamy paste with a weak, nutty roasted odor.

When the precursor is boiled in water, a nutty, roasted aroma (the aroma of acetylpyrazine) is released.

Upon tasting of the precursor (10 ppm) in an aqueous 5% sugar solution at room temperature, a weak, bland aroma that slowly develops a roasted bread crust aroma is perceived.

### Example 6

### (2-methyl-2-(3-methylpyridin-2-yl)-1,3-dioxolan-4-yl)methyl stearate

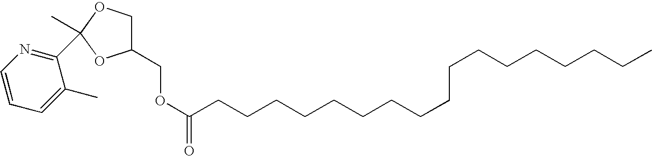

The precursor compound is a compound of formula I with n=1, m=0, R1=heptadecyl, R2=methyl, R3=3-methylpyridin-2-yl that is able to release 2-acetyl-3-methylpyridine.

The precursor is prepared using Glycerin-monostearate and 2-acetyl-3-methylpyridine as described in example 1. The purified precursor is a creamy paste with a weak, nutty roasted odor.

When the precursor is boiled in water, a nutty, roasted aroma (the aroma of 2-acetyl-3-methylpyridine) is released.

Upon tasting of the precursor (1 ppm) in an aqueous 5% sugar solution at room temperature, a weak, bland aroma that slowly develops a roasted nutty aroma is perceived.

### Example 7

### (2-methyl-1,3-dioxolan-4-yl)methyl decanoate

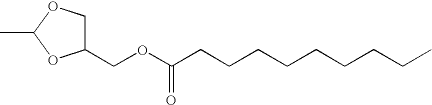

The precursor compound is a compound of formula I with n=1, m=0, R1=nonyl, R2=H, R3=methyl that is able to release acetaldehyde.

The precursor is prepared using Glycerin-monodecanoate as described in example 1 subject to the following modification:

5 g of cold (ca 10° C.) Acetaldehyde (CAS 75-07-0 Supplier: Aldrich) are dissolved in 20 ml cold hexane (ca 10° C.) with 5 g of glycerin monodecanaoate and a trace of HCl conc. as catalyst in a pressure reaction vessel and heated for ca. 2 hours. The purified precursor is a paste with a weak, chemical odor.

When the precursor is boiled in water, a fresh juicy, watery aroma (the aroma of acetaldehyde) is released.

Upon tasting of the precursor in an aqueous 20 ppm sugar solution at room temperature, a weak, bland aroma that slowly develops a fresh juicy, watery aroma is perceived.

### Example 8

### (2-acetyl-2methyl-1,3-dioxolan-4-yl)methyl dec-9-enoate

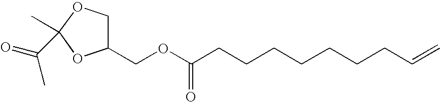

The precursor compound is a compound of formula I with n=1, m=0, R1=8-nonenyl, R2=methyl, R3=acetyl that is able to release diacetyl.

The precursor is prepared using 1-Glycerindec-9-enoate and diacetyl as described in example 1 subject to the following modification: 1 g of Glycerin-monodecanoate is replaced with 1 g 1-Glycerindec-9-enoate, which is synthesized as described below and reacted with 500 mg of diacetyl (CAS 431-03-8, Aldrich) as described in example 1. The purified percursor is a yellowish paste with a weak, buttery odor.

When the precursor is boiled in water, a fresh, buttery, creamy, fruity aroma (the aroma of diacetyl) is released.

Upon tasting of the precursor (1 ppm) in an aqueous 5% sugar solution at room temperature, a weak, bland aroma that slowly develops a fresh, buttery, creamy, fruity aroma is perceived.

Synthesis of 1-Glycerindec-9-enoate:

10 g of 9-Decenoic acid (CAS 14436-32-9, Bedoukian), 10 g of glycidol (CAS 556-52-5, Aldrich) and 100 mg Amberlyst A-26(OH) (Aldrich) are boiled under reflux with 200ml hexane in a round bottom flask with reflux condensor and magnetic stirrer for 24 hrs. The solution is filtered and the volatiles are distilled off under vacuum. The formed 1-Glycerindec-9-enoate can be used as such or purified further by chromatography.

### Example 9

### (2-heptyl-2-methyl-1,3-dioxolan-4-yl)methyl octanoate

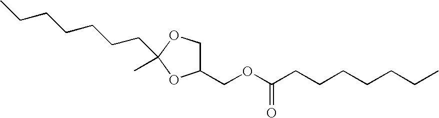

The precursor compound is a compound of formula I with n=1,m=0, R1=heptyl, R2=methyl, R3=heptyl (C7 saturated) that is able to release 2-nonanone.

The compound is prepared using Glycerin-monooctanoate and 2-nonanone as described in example 1 subject to the following modifications: 1 g of Glycerin-monooctanoate (CAS 502-54-5, also known as 1-Monocaprylin, commercially available from Indofine) and 500 mg of 2-nonanone (CAS 821-55-6, Aldrich) are dissolved in 20 ml hexane.

The resulting precursor material may be further purified by chromatography. The purified precursor is a whitish paste with a weak, ketony aroma.

When the precursor is boiled in water, a fresh, bluecheese, creamy, fruity aroma (the aroma of 2-nonanone) is released.

Upon tasting of the precursor (1 ppm) in an aqueous 5% sugar solution at room temperature, a weak, bland aroma that slowly develops a fresh, bluecheese, creamy, fruity aroma is perceived.

### Example 10

### (2-isobutyl-1,3-dioxolan-4-yl)methyl octanoate

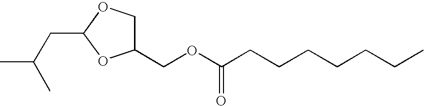

The precursor compound is a compound of formula I with n=1, m=0, R1=heptyl, R2=H, R3=2-methylpropyl that is able to release isovaleraldehyde.

The compound is prepared using Glycerin-monooctanoate and isovaleraldehyde as described in example 1 subject to the following modifications: 1 g of Glycerin-monooctanoate (CAS 502-54-5, also known as 1-Monocaprylin, commercially available from Indofine) and 500 mg of isovaleraldehyde (CAS 590-86-3, Aldrich) are dissolved in 20 ml hexane. The purified precursor is a whitish paste with a weak, aldehydic aroma.

When the precursor is boiled in water, a fresh, cocoa aroma (the aroma of isovaleraldehyde) is released.

Upon tasting of the precursor (1 ppm) in an aqueous 5% sugar solution at room temperature, a weak, bland aroma that slowly develops a cocoa aroma is perceived.

### Example 11

### (2-isobutyl-1,3-dioxan-5-yl)laurate

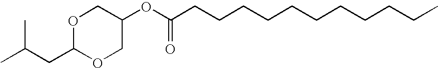

The precursor compound is a compound of formula I with n=0, m=1, R1=undecyl, R2=H, R3=2-methylpropyl that is able to release isovaleraldehyde.

The compound is prepared using Glycerin-2-monododecanoate and isovaleraldehyde as described in example 1 subject to the following modifications: 1 g of Glycerin-2-monododecanoate (CAS 1678-45-1, also known as 2-Monolaurin, commercially available from Indofine) and 500 mg of isovaleraldehyde (CAS 590-86-3 Supplier: Aldrich) are dissolved in 20 ml hexane. The purified precursor is a whitish paste with a weak, aldehydyc aroma.

When the precursor is boiled in water, a fresh, cocoa aroma (the aroma of isovaleraldehyde) is released.

Upon tasting of the precursor (1 ppm) in an aqueous 5% sugar solution at room temperature, a weak, bland aroma that slowly develops a cocoa aroma is perceived.

### Example 12

### Cocoa Flavor Precursor

Preparation of Cocoa-Monoglyceride:

A mixture of monoglycerides, derived from cocoabutter is prepared by stirring 60 g cocoabutter with 20 g glycerine, catalyzed by 100 mg HCl conc. in a roundbottom flask at 100C for 20 hours.

For the topnote, 1.0 g Isovaleraldehyde, 5.0 g 2-Methylbutyraldehyde, and 1.0 g Phenylacetaldehyde are mixed together.

For the top aroma, 1 g topnote are mixed with 99 g Migliol (vegetable oil). The top aroma has a very strong fresh cocoa aroma.

Preparation of the Precursors:

1 g topnote is mixed with 20 g of Cocoa-Monoglyceride and heated for 10 hours in a roundbottom flask with reflux cooler to form the precursors. The resulting cooled precursor mixture has a weak cocoa aroma.

Comparison of Precursors and Top Aroma in a Milk Drink:

Top aroma: A milk-drink powder is flavored with 0.1% (wt/wt) of the top aroma.

Precursor mixture: A milk-drink powder is flavored with 0.1% (wt/wt) of the Precursor mixture.

From the freshly prepared milk drink power, hot milk drinks are prepared and sensorically evaluated. The top aroma flavored milk drink has a strong, pungent cocoa aroma, while the precursor mixture flavored milk drink has a weaker balanced cocoa aroma.

After 2 months storage at room temperature of the drink powder, a sensory evaluation is performed by adding the powder to hot milk and stirring for 2 minutes. The top aroma milk drink is bland and has completely lost its original cocoa aroma. The precursor mixture milk drink shows a nice, balanced cocoa aroma and has a good mouthfeel.

### Example 13

### Apple Flavor Precursor

Preparation of Apple-Monoglyceride:

A mixture of 25 g Triacetin, 25 g Miglyol, 20 g glycerin, 25g 2-methylbutyric acid and 100 mg HCl conc. is stirred in a roundbottom flask with distillation head at 110° C. for 6 hours. The formed water is distilled off. The formed product is used without further purification.

For the topnote, 5.0 g hexanal and 5.0 g 2-hexenal are mixed together.

For the top aroma, 1 g topnote are mixed with 99 g migliol (vegetable oil). This resulting top aroma has a very strong fresh apple aroma.

Preparation of the Precursors:

1 g of topnote is mixed with 20 g of apple-monoglyceride and heated for 8 hours in a roundbottom flask with reflux cooler to form the precursors. The resulting cooled mixture has a weak apple aroma.

Comparison of Precursors and Top Aroma in a biscuits:

A biscuit short dough is prepared as follows:

| Ingredients: | % (wt/wt) |
|---|---|
| 1) Plain Flour (~10% (wt/wt) protein level) | 52.31 |
| 2) Vegetable Shortening BM 3030 | 17.26 |
| (Woodlands Sunny Foods, Senoko, Singapore) | |
| 3) Fine Milled Sugar | 17.40 |
| 4) Glucose Syrup 42 DE | 3.45 |
| 5) Skim Milk Powder | 1.49 |
| 6) Salt | 0.25 |
| 7) Sodium Bicarbonate | 0.31 |
| 8) Ammonium Bicarbonate | 0.21 |
| 10a) top aroma | 0.2 |
| 10b) precursor | 0.1 |
| | add 100.00 (water) |

Ingredients 2-6 are pre-blended in the mixing bowl, and mixed at low speed, then mixed further at medium speed for about 3 minutes. At low speed, the pre-dissolved solutions of sodium bicarbonate, ammonium carbonate, and 0.2% (wt/wt) of the top aroma or 0.1% (wt/wt) of the precursors are added; then the remaining water is added and mixed for 1 minute. Mixing is continued at medium speed for about 3 minutes to form a homogenous mixture. The flour is added at low speed to obtain a dough. The dough is formed to a sheet of a thickness of 4 mm and cut into biscuit shapes using a stamp cutter. Biscuits are baked on a wire tray at a temperature of 200° C. for about 8 to 10 minutes.

During the baking process a strong apple aroma from the baking oven is observed from the top aroma biscuits while the precursor biscuits develop a weaker aroma during baking. On sensory evaluation of the fresh biscuits, the precursor biscuits have a weaker aroma than the top aroma biscuits.

After 2 months of storage at room temperature a comparative sensory evaluation is performed. The top aroma biscuits are bland and have completely lost their original apple aroma. The precursor biscuits show a nice, balanced fruity apple aroma.

### Example 14

### Potato Flavor Precursor

Preparation of Potato-Monoglyceride:

A mixture of monoglycerides derived from peanut oil is prepared by stirring 60 g peanut oil with 20 g glycerine, 1 g of methionine, catalyzed by 100 mg HCl conc. in a roundbottom flask at 100° C. for 20 hours.

For the topnote, 0.05 g Methional, 1.0 g 2-Methyl-2-(methyldithio)propanal (FEMA3866), and 0.1 g 3-Methylthiobutanal (FEMA3374) are mixed.

For the top aroma, 1 g topnote is mixed with 99 g peanut oil. The top aroma has a very strong potato aroma.

Preparation of the Precursors:

1 g of topnote is mixed with 20 g of Potato-Monoglyceride and heated for 10 hours in a roundbottom flask with reflux cooler to form the precursor. The resulting cooled mixture has a weak potato aroma.

Comparison of Precursors and Top Aroma in Potato Flakes/Mashed Potato:

Potato flakes are flavored with 0.1% (wt/wt) of the top aroma or the precursors and evaluated sensorically. The top aroma flakes (without cooking/heating) have a stronger potato aroma than the precursor.

After 2 months of storage at room temperature of the flavored flakes a comparative sensory evaluation is performed. The samples are cooked in water to mashed potato and compared. The top aroma sample is bland, has completely lost its original potato aroma and shows a starchy, watery aroma. The prescursor sample shows a nice, balanced potato aroma.

### Example 15

### Cheese Flavor Precursor

Preparation of Cheese-Monoglyceride:

A mixture of 25 g Miglyol, 25 g Tripropionin, 20 g glycerin, 25 g butyric acid and 100 mg HCl conc. is stirred in a roundbottom flask with distillation head at 100° C. for 6 hours. The formed water is distilled off. The formed product is used without further purification.

For the topnote, 2 g 2-Heptanone (FEMA2544), 0.5 g 2-Nonanone FEMA2785), 1.0 g 2-Octanone (FEMA2802) 2.0 g 2-Undecanone (FEMA3093), 2.0 g Diacetyl, and 0.5 g 2-Methyl-2-(methyldithio)propanal (FEMA3866) are mixed.

For the top aroma, 1 g topnote are mixed with 99 g miglyol (vegetable oil). The top aroma has a very strong blue cheese aroma.

Preparation of the Precursors:

1 g of topnote is mixed with 20 g of Cheese-Monoglyceride and heated for 10 hours in a roundbottom flask with reflux cooler to form the precursor. The resulting precursors have a weak blue cheese aroma.

Comparison of Precursors and Top Aroma in Cracker Biscuits:

A standard dough for crackers is prepared as follows:

| Ingredients: | % Weight |
|---|---|
| 1) Plain Flour (about 10% (wt/wt) protein) | 59.50 |
| 2) Monocalcium Phosphate | 0.60 |
| 3) Vegetable Shortening BM3030 | 7.14 |
| (Woodlands Sunny Foods, Senoko, Singapore) | |
| 4) Fine Milled Sugar | 2.23 |
| 5) Salt | 0.89 |
| 6) Glucose Syrup 42DE | 4.76 |
| 7) Sodium Bicarbonate | 0.60 |
| 8) Ammonium Bicarbonate | 2.23 |
| 9) Sodium Metabisulphite | 0.02 |
| 10a) top aroma | 0.2 |
| 10b) precursor | 0.2 |
| | add 100.00 (water) |

Ingredients 1 and 2 are sieved and dry-blended in a bowl. Ingredients 3 to 6 are placed in a mixing bowl, added to the above dry-blend and mixed at low speed for about 1 minute. The pre-dissolved solutions of sodium bicarbonate, ammonium carbonate and sodium metabisulphite are added at low speed. The top aroma or precursor is added. The remaining water is added and the cracker dough is mixed at medium speed at a temperature of 28-30° C. for about 5 minutes until gluten development is achieved. The dough is left to rest at 28-30° C. for 5 minutes. The dough is formed into a sheet and laminated twice until a final thickness of 1.5 mm is obtained. The dough sheet is cut into cracker shapes and lightly dusted with fine salt. The cracker dough is baked at 230° C. for about 5-7 minutes. Baked crackers are brushed with warm oil (˜80° C.).

During the baking process a strong blue cheese aroma is released from the baking oven for the top aroma crackers but not the precursor crackers. The resulting fresh biscuits with top aroma have a stronger flavor than the precursor crackers.

After 2 months storage at room temperature a comparative sensory evaluation is performed. The top aroma crackers are bland and have completely lost its original blue cheese aroma. The precursor crackers show a nice, balanced cheese aroma.

### Example 16

### Butter Flavor Precursor

Preparation of Butter-Monoglyceride:

A mixture of monoglycerides, derived from butter fat is prepared by stirring 60 g butter fat with 20 g glycerine, catalyzed by 600 mg of Lipozyme RM IM (Novozyms) in a roundbottom flask at 50° C. for 24 hours.

For the topnote, 2.0 g Diacetyl and 0.001 g cis-4-Heptenal are mixed.

For the top aroma, 1 g topnote are mixed with 99 g miglyol (vegetable oil). This flavor has a very strong butter aroma

Preparation of the Precursors:

1 g of top note is mixed with 20 g of Butter-Monoglyceride and heated for 10 hours in a round bottom flask with reflux cooler to form the precursor. The resulting cooled precursor mixture has a weak butter aroma.

Comparison of Precursors and Top Aroma in Biscuits:

A standard dough for biscuit is prepared as in example 13.

0.2% of the top aroma or 0.1% of the precursors are homogenously mixed with the dough. Biscuits are formed and baked at an oven temperature of 200° C. for 20 min.

During the baking process a strong butter aroma from the baking oven is observed for the top aroma biscuits, but which is very weak for the precursor biscuits. The sensory evaluation shows a stronger aroma in the top aroma biscuits.

After 2 months storage at room temperature a sensory evaluation is performed. The top aroma sample is bland, has completely lost its original butter aroma, and has a dry texture and mouth feel. The precursor biscuits show a nice, balanced butter aroma and smooth, lasting buttery mouth feel.

### Example 17

### Bread Flavor Precursor

Preparation of Bread-Monoglycerides:

A mixture of monoglycerides derived from butter fat is prepared by stirring 60 g butter fat, 2 g proline with 20 g glycerine, catalyzed by 600 mg of Lipozyme RM IM (Novozyms) in a roundbottom flask at 50° C. for 24 hours.

For the topnote, 2 g Acetylpyrazine, 1 g 2-Acetylpyridine, 0.5 g 2-Acetylthiazole, and 2 g Butyraldehyde are mixed.

For the top aroma, 1 g topnote are mixed with 99 g peanut oil. This top aroma has a very strong roasted aroma.

Preparation of the Precursors:

1 g of topnote is mixed with 20 g of bread-Monoglycerides and heated for 10 hours in a round bottom flask with reflux cooler to form the precursor. The resulting cooled precursors have a weak roasted aroma.

Comparison of Precursors and Top Aroma in Bread Buns:

A standard dough for bread bun is prepared as follows:

| Ingredients | % Weight |
|---|---|
| 1) Bread Flour | 53.81 |
| 2) Vegetable Shortening BM 3030 | 3.77 |
| (Woodlands Sunny Foods, Senoko, Singapore) | |
| 3) Castor Sugar | 5.92 |
| 4) Skim Milk Powder | 1.13 |
| 5) Salt | 1.21 |
| 6) Instant Yeast | 0.81 |
| 7a) top aroma | 0.2 |
| 7b) precursor | 0.2 |
| | add 100.00 (water) |

The instant yeast is hydrated using part of the water. At low speed, ingredients 1 to 4 and the remaining water are mixed in a mixing bowl. Salt is added. The dough is mixed at medium speed for about 10 minutes to form a soft and shiny dough with a temperature of about 24 to about 27° C.

The dough is formed into 65 g buns (determined with a scale, moulded, and the pieces are panned. The buns are set in a proofer for about 60 minutes at relative humidity 85 to 90% and temperature 43 to 46° C until proof. Then the buns are baked at 230° C. for about 7 minutes.

0.2% (wt/wt) of the top aroma or precursors are homogenously mixed with the dough. Buns are formed and baked at an oven temperature of 200° C. for 20min.

During the baking process a strong roasted aroma from the baking oven is observed for the top aroma buns which is much weaker for the precursor buns. When evaluated sensorically directly after baking, the top aroma buns have a stronger roasted bread aroma than the precursor buns.

After 2 weeks storage at room temperature a sensory evaluation is performed. The top aroma version is bland and has lost its original roast aroma and has a starchy, dry “retrograded” aroma.

The precursor buns show a nice, balanced roasted aroma.

### Example 18

### Hazelnut Flavor Precursor

Preparation of Hazelnut-Monoglycerides

A mixture of 50 g Miglyol, 20 g glycerin and 100 mg HCl conc. is stirred in a roundbottom flask with distillation head at 110° C. for 6 hours. The formed water is distilled off. The formed product is used without further purification.

For the topnote, 1.0 g 2-Acetyl-3-ethylpyrazine, 1 g 2-Acetyl-3-methylpyrazine, 0.5 g 5-Methyl-hept-2-en-4-one (FEMA3761), 1.0 g Isovaleraldehyde are mixed.

For the top aroma, 1 g topnote are mixed with 99 g peanut oil. The top aroma has a very strong roasted nutty aroma.

Preparation of the Precursors:

1 g of topnote is mixed with 20 g of Hazelnut-Monoglyceride and heated for 10 hours in a roundbottom flask with reflux cooler to form the precursor. The resulting cooled mixture has a weak roasted, nutty aroma.

Comparison of Precursors and Top Aroma in Chocolate:

A Chocolate Mass is Prepared as Follows:

| Ingredients: | % Weight |
|---|---|
| 1) Compound dark super-coat chocolate | 92.59 |
| (Barry Callebaut Asia Pacific, Singapore) | |
| 2) Palm Kernel Stearin (Barry Callebaut Asia | 7.41 |
| Pacific, Singapore) | |
| 3a) top aroma, or alternatively precursor) | 0.2 |
| | add 100.00 (water) |

Compound dark super-coat chocolate and stearin are filled into a beaker and melted and stirred over a pot of warm water (about 35 to 38° C.) until the chocolate mass is smooth and uniform (free of lumps). The top aroma or precursor are added. The resulting mass has a roasted nutty hazelnut chocolate aroma.

The chocolate mass is coated onto the nutritional bar product.

Nutritional Bars are Prepared as Follows:

| Ingredients Binder syrup: | % Weight |
|---|---|
| 1) Isomalt (Palatinit Asia Pacific, Singapore) | 35.81 |
| 2) Sugar | 18.00 |
| 3) Dextrose | 4.40 |
| 4) Glucose Syrup 42 DE | 15.00 |
| 5) Condensed Milk | 2.50 |
| 6) Glycerine 85%, E-009 | 3.00 |
| 7) Salt | 0.20 |
| 8) Vegetable Shortening BM 3030 | 10.00 |
| (Woodlands Sunny Foods, Senoko, Singapore) | |
| 9) Lecithin, Topcithin ™ N50 (Degussa texturants | 0.08 |
| systems, Singapore) | |
| | add 100.00 (water) |

| Ingredients nutritional bar: | % Weight |
|---|---|
| 1) Corn Flakes (Crushed) | 8.00 |
| 2) Rice Krispies | 10.71 |
| 3) Rolled Oats | 24.11 |
| 4) Binder Syrup (as above) | 57.18 |
| | add 100.00 (water) |

Ingredients 1 to 7 and water are mixed in a mixing bowl. The vegetable shortening is melted and stirred into the lecithin, then added to the mixing bowl and mixed at low speed for 1 minute or until homogenous. The mixture is further mixed at medium speed for 2 minutes until the mixture is almost opaque. The mixture is transferred to a pot and heated to 120° C. (84.8° Brix) upon continuous stirring.

The cooked syrup is poured on top of the dry cereal mix and stirred gently ensuring that the binding syrup covers the cereals thoroughly. The syrup-cereal mix is emptied into a mould which is levelled out with a scrapwer. The syrup-cereal mixture is packed tightly, and rolled out with a rolling pin. The syrup-cereal mixture is cooled and cut into bars measuring 10×3.0×1.5 cm. The bars are wrapped and packed.

The nutritional bars are coated with the chocolate mass and evaluated sensorically. The top aroma bar shows a stronger nutty aroma than the precursor bar.

After 2 months storage at room temperature a second sensory evaluation of the bars is performed. The top aroma bar has a bland neutral chocolate cover and has completely lost its original hazelnut aroma. The precursor bar shows a nice, balanced hazelnut-chocolate aroma.

### Example 29

### Tomato Flavor Precursor

Preparation of Tomato-Monoglyceride:

A mixture of 25 g Tripropionin, 25 g Miglyol, 20 g glycerin, 25 g 3-Methylbutyric acid and 100 mg HCl conz. is stirred in a roundbottom flask with distillation head at 110° C. for 6 hours. The formed water is distilled off. The formed product is used without further purification.

For the topnote, 0.05 g Methional, 1.0 g 2,4-Hexadienal (FEMA3429), 0.5 g cis-3-Hexenal, and 0.1 g Isobutyl thiazole (FEMA3134) are mixed.

For the top aroma, 1 g topnote are mixed with 99 g peanut oil. The top aroma has a very strong tomato aroma

Preparation of the Precursors:

1 g of topnote is mixed with 20 g of tomato-monoglyceride and heated for 4 hours in a round bottom flask with reflux cooler to form the precursor. The resulting cooled precursor mixture has a weak tomato aroma.

Comparison of Precursors and Top Aroma in Tomato Soup:

A Tomato Cream Soup is Prepared as Follows:

| Ingredients Instant cream soup base | % (wt/wt) |
|---|---|
| Salt | 12.00 |
| Sugar | 8.50 |
| monosodiumglutamate | 1.50 |
| I + G (inosine monophosphate•guanosine | 0.10 |
| monophosphate; nucleotides) | |
| Palm fat | 5.00 |
| Chicken Roast Flavor 605-00015-53 (Givaudan, | 5.00 |
| Cincinnati, USA) | |
| Onion powder | 1.00 |
| Garlic powder | 0.80 |
| Novation 5600 (ex. National Starch) | 42.00 |
| Non-dairy creamer | 15.55 |
| Skimmed milk powder | 7.95 |
| Butter Flavor CF 8.08.07.L (Givaudan, Cincinnati, | 0.15 |
| USA) | |
| Vegetable Stock 473869 (Givaudan, Cincinnati, | 0.50 |
| USA) | |
| | Add 100.00 (water) |

Palm fat is melted and plated into salt, sugar, monosodiumglutamate and I+G. The rest of the ingredients is added and mixed until homogenous. The mixture is sieved and packed.

| Ingredients tomato soup powder | % (wt/wt) |
|---|---|
| Instant Cream Soup Base | 76.80 |
| Tomato powder (Spreda 707) | 20.00 |
| Citric acid | 0.40 |
| Lycopene 10% ws (Roche) | 0.40 |
| Parsley flakes | 0.40 |
| Ultra Rhodigel ™ (Rhodia) | 1.00 |
| top aroma or precursor | 0.1% (wt/wt)? |
| | Add 100.00 (water) |

To prepare the tomato soup, 6.5 g of the tomato soup powder is used and added to 100 ml hot boiling water. The soup is stirred and served in cups for sensorical evaluation,

The top aroma soup has a strong, pungent tomato aroma stronger than the precursor soup.

After 3 months storage at room temperature of the soup powder samples, a second evaluation is performed with prepared heated soup samples. The top aroma soup is bland and has completely lost its original tomato aroma. The precursor soup shows a nice, balanced tomato aroma.

1. A method of providing a flavored food product, wherein at least one flavor precursor of formula I 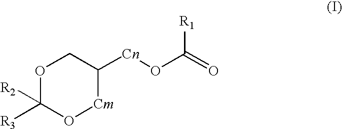
wherein n and m are selected from 0 and 1, and if n is 1 then m is 0, and if n is 0 then m is 1;
wherein R1 is selected from the group consisting of
a straight-chain C7 to C17 alkyl, a straight-chain C7 to C17 alkenyl, a branched C7 to C17 alkyl, a branched C7 to C17 alkenyl,
a straight-chain C7 to C17 monoalkenyl, a branched C7 to C17 monoalkenyl,
a straight-chain C7 to C17 alkadienyl, a branched C7 to C17 alkadienyl,
a straight-chain C7 to C17 alkatrienyl, a branched C7 to C17 alkatrienyl,
a straight-chain C9 to C17 alkatetraenyl, a branched C9 to C17 alkatetraenyl,
and a straight-chain C11 to C17 alkapentaenyl, a branched C11 to C17 alkapentaenyl, and
wherein the R2-CO-R3 part of formula I is the residue of a flavor compound, wherein the O of the R2-CO-R3 is the remainder of the reacted carbonyl group of said flavor compound,
and wherein R2-CO-R3 has a molecular weight from 44 to 294;
is admixed to a food product in a sufficient concentration to release a flavor of noticeable aroma upon consumption and/or heating of the food product.

2. The method according to claim 1 wherein said flavor is selected from the group consisting of:
(+)-8,9-DEHYDRONOOTKATONE; (1R-CIS AND TRANS)-2-(1-ACETYLTHIO-1-METHYL)ETHYL-5-METHYLCYCLOHEXANONE; (E)-2-(2-OCTENYL)CYCLOPENTANONE; (E)-5-ISOPROPOXY-2-DECENAL; (E,E)-3,5-OCTADIEN-2-ONE; (METHYLTHIO)ACETONE; (RAC)-3-ACETYLOXY-5-METHYL-2-HEXANONE; 1-(2,5,5-TRIMETHYL-CYCLOPENT-1-EN-1-YL)-3-METHYL-2-BUTEN-1-ONE; 1-(3,5,5,-TRIMETHYL-1-CYCLOHEXENYL)-3-METHYL-2-BUTEN-1-ONE; 1(5-METHYLFURYL-2)-PROPANE-1,2-DIONE; 1-(METHYLTHIO)-2-BUTANONE; 1-(METHYLTHIO)-3-PENTANONE; 1-(METHYLTHIO)-OCTAN-3-ONE; 1-(P-METHOXYPHENYL)-2-PROPANONE; 1,4-DODEC-6-ENOLACTONE; 1,5,5-TRIMETHYL-8-ETHYL-G-OXA-BICYCLO(4,3,O)NON-6-en-3-one; 1,5-[Z]-OCTADIEN-3-ONE; 10-UNDECENAL; 12-METHYL-TRIDECANAL; 14-METHYL-PENTADECANAL; 1-HYDROXY-2-BUTANONE; 1-MERCAPTO-2-PROPANONE; 1-MERCAPTO-3-PENTANONE; 1-PENTEN-3-ONE; 2-(3,3-DIMETHYLCYCLOHEXYLIDEN)-ETHANAL; 2-(METHYLTHIO)METHYL-2-BUTENAL; 2-(METHYLTHIOMETHYL)-3-PHENYLPROPENAL; 2,10-UNDECADIENAL; 2,3-DIHYDRO-2,3,3-TRIMETHYL-1H-INDEN-1-ONE; 2,4-DIMETHYL-HEPT-4-ENE-3-ONE; 2,4-PENTADIENAL; 2,5-DIETHYL-4-HYDROXY-5-METHYL-3(2H)-FURANONE; 2,5-DIMETHYL-3 (2H)-FURANONE; 2,5-DIMETHYL-4-HYDROXY-6-HEPTANONE; 2,5-DIMETHYL-TETRAHYDRO-3-FURANONE; 2,6-DIMETHYL-3-HYDROXY-4-PYRONE; 2,6-DIMETHYL-4-HEPTANONE; 2,6-DIMETHYLBICYCLO[3.2.1 ]OCT-2-EN-7-ONE; 2-AMINO-ACETOPHENONE; 2-BUTYL-2-BUTENAL; 2-ETHYL-4-METHYL-PENT-2-ENAL; 2-HEPTEN-4-ONE; 2-HEPTYL-BUTYROLACTONE; 2-Hexenal; 2-HYDROXY-1-(4-HYDROXY-3-METHOXY-PHENYL)-ETHANONE; 2-HYDROXY-2-CYCLOHEXEN-1-ONE; 2-HYDROXY-3,4,5-TRIMETHYL-2-CYCLOPENTEN-1-ONE; 2-HYDROXY-3,5,5-TRIMETHYL-2-CYCLOHEXENONE; 2-HYDROXYACETOPHENONE; 2-ISOPROPYL-5-CAPROLACTONE; 2-ISOPROPYL-DIOXOLAN-4-ONE; 2-METHOXY-3,4,5-TRIMETHYL-2-CYCLOPENTEN-1-ONE; 2-METHYL-2-OCTENAL; 2-METHYL-3-(P-METHYLPHENYL)PROPANAL; 2-METHYL-8-PHENYL-OCT-2-ENE-6-ONE; 2-METHYLCYCLOHEXANONE; 2-METHYLHEPTAN-3-ONE; 2-METHYLHEXAN-3-ONE; 2-METHYLNONANAL; 2-METHYL-SPIRO(5,5)-UNDECAN-1-ONE; 2-OCTEN-4-ONE; 2-PHENYL-3-(2-FURYL)PROP-2-ENAL; 2-PHENYL-4-PENTENAL; 2-TETRADECENAL; 2-TRANS-4-CIS-7-CIS-TRIDECATRIENAL; 3-((2-METHYL-3-FURYL)THIO)-4-HEPTANONE; 3(2)-HYDROXY-4-METHYL-HEXAN-2(3)-ONE; 3-(3-METHOXY-PHENYL)-PROPAN-2-ONE; 3-(5-METHYL-2-FURYL)BUTANAL; 3(Z),6(Z),9(Z)-DODECATRIENAL; 3(Z),6(Z)-DODECADIENAL; 3,3-DIETHOXY-2-BUTANONE; 3,3-DIMETHYLCYCLOHEXANONE; 3,4,5,6-TETRAHYDRO PSEUDO IONONE; 3,4-DIMETHYL-BUTYROLACTONE; 3,5-DIMETHYL-4-THIA-HEPTAN-2,6-DIONE; 3,6,DIMETHYL-5,6-DIHYDRO-2(4H)BENZOFURANONE; 3,6-DIMETHYL-2-HYDROXY-2-CYCLOHEXEN-1-ONE; 3,7-DIMETHYL-6-OCTEN-2-ONE; 3A,4,5,7A-TETRAHYDRO-3,6-DIMETHYL-2(3H)BENZOFURANONE; 3-ACETYLOXY-2-OCTANONE; 3-ACETYLOXY-2-PENTANONE; 3-ACETYLSULFANYL-2-ETHYLHEXANAL; 3-DECANONE; 3-ETHOXY HEXANAL; 3-ETHYL-2-HYDROXY-4-METHYLCYCLOPENT-2-EN-1-ONE; 3-ETHYL-4,5,5-TRIMETHYL-2-(5H)-FURANONE; 3-HEPTYLDIHYDRO-5-METHYL-2(3H)-FURANONE; 3-HEXANONE; 3-HYDROXY-2-PENTANONE; 3-HYDROXY-4,5,5-TRIMETHYL-2-(5H)-FURANONE; 3-HYDROXY-4-METHYL-5-PENTYL-2(5H)-FURANONE; 3-HYDROXY-4-METHYL-ALPHA-PYRONE; 2-ACETYL-4,5-DIMETHYL-THIAZOLE; 2-METHYL-3-ACETYLPYRIDINE; 1-ETHYL-2-ACETYL-PYRROLE; 1-METHYL-2-ACETYLPYRROLE; 2,4-DIMETHYL-5-ACETYL THIAZOLE; 3-ACETYL-2,5-DIMETHYLFURAN; 1,4-DIMETHYL-4-ACETYL-1-CYCLOHEXENE; 4-ACETYL-6-T-BUTYL-1,1-DIMETHYLINDAN; 4-ACETYL-2-METHYLPYRIMIDINE; ACETYL-2-PYRAZINE; ACETYL-2-FURAN; ACETYL-2-PYRROLE; ACETYL-2-PYRIDINE; ACETYL-2-DIMETHYL-PYRAZINE; ACETYL-2-THIAZOLE; ACETYL-3 PYRIDIN; ACETYL-3-DIMETHYL-2.5-THIOPHENE; ACETYL-2 METHYL-5 FURAN; ACETYL-2-THIAZOLINE-2; ACETYL-2 METHYL-3 PYRAZINE; PROPIONYL PYRAZINE; PROPIONYL-2-METHYL-3-FURANE; 2-PROPIONYLTHIAZOLE; 2-PROPIONYLPYRROLE; 3-HYDROXY-4-PHENYL-2-BUTANONE; 3-HYDROXY-5-HEXYL-4-METHYL-2(5H)-FURANONE; 3-HYDROXY-6-HYDROXYMETHYL-PYRAN-2-ONE; 3-HYDROXYMETHYL-2-OCTANONE; 3-ISOAMYL-2,4-PENTANDIONE; 3-MERCAPTO-2-METHYLPENTANAL; 3-MERCAPTO-2-OCTANONE; 3-MERCAPTO-2-PENTANONE; 3-MERCAPTO-4-PHENYL-2-BUTANONE; 3-MERCAPTO-5-METHYL-HEXAN-2-ONE; 3-METHYL NON-2(E)-EN-4ONE; 3-METHYL-1-CYCLOPENATADECANONE; 3-METHYL-2-CYCLOHEXEN-1-ONE; 3-METHYL-2-CYCLOPENTEN-1-ONE; 3-METHYL-2-HEPTEN-4,5-DIONE; 3-METHYL-3-(3-METHYL-BUT-2-ENYL)-DIHYDRO-FURAN-2-ONE; 3-METHYL-4-PHENYL-3-BUTENE-2-ONE; 3-METHYL-5-PROPYL-2-CYCLOHEXEN-1-ONE; 3-METHYLCYCLOHEXANONE; 3-METHYLENE-2-OCTANONE; 3-METHYL-GAMMA-DECALACTONE; 3-METHYLPENTANAL; 3-METHYLTHIOHEXANAL; 3-NONANONE; 3-NONEN-2-ONE; 3-PENTANONE; 3-PENTEN-2-ONE; 3-PENTYL-4,5,5-TRIMETHYL-2-(5H)-FURANONE; 3-PHENYL-4-PENTENAL; 3-PROPYL-2-CYCLOPENTEN-1-ONE; 3-PROPYLIDEN-HEPTAN-2-ONE; 4-(2,6,6-TRIMETHYL-2-CYCLOHEXEN-1-YL)BUTAN-2-ONE; 4-(2,6,6-TRIMETHYL-CYCLOHEXA-1,3-DIENYL)-BUT-3-EN-2-ONE; 4-(FURAN-2-YL)-BUTAN-2-ONE; 4-(METHYLTHIO)BUTANAL; 4-(P-TOLYL)-2-BUTANONE; 4,4-DIMETHOXY-3-HEXANONE; 4,4-DIMETHYL-1,3-OXATHIANE-2-ONE; 4,8-DIMETHYL-3,7-NONADIEN-2-ONE; 4-[(2-FURANMETHYL)THIO]-2-PENTANONE; 4-ACETOXY-HEX-4-EN-3-ONE; 4-EPOXY-1,1,3-TRIMETHYLCYCLOHEX-5-EN-2-ONE; 4-ETHYL-1,3-OXATHIAN-2-ONE; 4-ETHYL-5-METHYL-1,3-OXATHIAN-2-ONE; 4-HEPTYL-3-METHYLBUTYROLACTONE; 4-HYDROXY-2,2,5-TRIMETHYL-3[2H]-FURANONE; 4-HYDROXY-3,5,5-TRIMETHYL-CYCLOHEX-5,5-EN-1-ONE; 4-HYDROXY-4-METHYL-5-HEXENOIC-ACID LACTONE; 4-HYDROXY-4-METHYL-CIS-7-DECANOICACID-4-LACTONE; 4-MERCAPTO-4-METHYL-PENTAN-2-ONE; 4-METHYL-2-PENTENAL; 4-METHYL-3-PENTEN-2-ONE; 4-METHYL-4-FURFURYLTHIO-2-PENTANONE; 4-METHYL-5-PENTYL-DIHYDRO-2(3H)-FURANONE; 4-METHYLCYCLOHEXANONE; 4-METHYLTHIO-2-PENTANONE; 4-OXO-BETA DAMASCONE; 4-PHENYL-3,5-DITHIA-2-HEXANONE; 5 ALPHA-ANDROST-16-EN-3-ONE; 5,5-DIMETHYL-2(5H)-FURANONE; 5,5-DIMETHYL-2-METHOXY-CYCLOHEX-2-ENONE; 5-ETHYL-2-HYDROXY-3,4-DIMETHYL-2-CYCLOPENTEN-1-ONE; 5-ETHYL-2-METHOXY-3,4-DIMETHYL-2-CYCLOPENTEN-1-ONE; 5-ETHYLDIHYDRO-5-METHYL-2(3H)-FURANONE; 5-HYDROXY-4-OCTANONE; 5-HYDROXY-8-UNDECENOIC ACID D-LACTONE; 5-METHYL-2-PROPYL-[1,3]-DIOXOLAN-4-ONE; 5-METHYL-3-(3-METHYL-BUT-2-ENYL)-DIHYDRO-FURAN-2-ONE; 5-METHYL-3-HEXEN-2-ONE; 5-METHYL-4-HEXENE-2,3-DIONE; 5-METHYL-5-HEXEN-2-ONE; 5-PENTYL-ALPHA-PYRONE; 5-P-TOLUYL-2(3H)-FURANONE; 6-(1-PENTENYL)-2H-PYRAN-2-ONE; 6-HEPT-1-ENYL-5,6-DIHYDRO-PYRAN-2-ONE; 6-HYDROXY-3,7-DIMETHYLOCTANOIC ACID LACTONE; 6-METHYL-3-HEPTEN-2-ONE; 6-METHYL-NON-5-EN-4-ONE; 6-METHYL-OCTANAL; 6-PROPYL-[1,3]OXATHIAN-2-ONE; 6-UNDECANONE; 7-METHYL-2,3,4,4A,5,6-HEXAHYDRO-2-NAPHTHALENONE; 7-METHYL-6-OCTEN-2-ONE; 7-OCTENAL; 8-(2,2-DIMETHYLCYCLOPROPYL)-OCTA-3,5-DIEN-2-ONE; 8A-METHYL-3,4,4A,5,8,8A-HEXAHYDRO-1(2H)-NAPHTALENONE; 8-METHYL NONANAL; 8-METHYL-TRIDEC-7-EN-6-ONE; 8-NONEN-2-ONE; 8-NONENAL; 9-DECENAL; ACETONE; ACETOPHENONE; A-P-DIMETHYLANISALACETONE; AR-TURMERONE; BENZOPHENONE; BENZOYLACETONE; BENZYL DIPROPYL KETONE; BENZYL-ACETONE; BENZYLIDENE ACETONE; BETA-IRONE; BICYCLO-IRONE; BICYCLONE; BICYCLONONALACTONE; BUTYRO-1,4-LACTONE; CARYOPHYLLEN-12-AL; CIS,CIS-4,7-DECADIEN-1-AL; CIS-2-NONENAL; CIS-3-HEXENAL; CIS-3-NONENAL; CIS-4-HEPTEN-2-ONE; CIS-4-HEXENAL; CIS-4-NONENAL; CIS-5-NONENAL; CIS-5-OCTENAL; CIS-7-NONENAL; CITRONELLENE LACTONE; CLONAL; COGNAC LACTONE; CYCLOBUTONE; CYCLOHEPTADECA-9-EN-1-ONE; CYCLOHEXANONE; CYCLOIONONE; CYCLOPENTANONE; CYLCOHEPTADECANONE; D-1-(2,6,6-TRIMETHYL-3-CYCLOHEXEN-1-YL)-2-BUTEN-1-ONE; DAMASCENONE; DECANAL; DECENYL CYCLOPENTANONE; DIBENZYL KETONE; DIBUTYL-4,4 GAMMA BUTYROLACTONE; DIHYDRO FILBERTONE; DIHYDRO-5-METHYL-5-PROPYL-2(3H)-FURANONE; DIHYDROCARVONE; DIHYDRO-DEHYDRO-GAMMA-IONONE; DIHYDROJASMONE LACTONE; DIHYDROLAVENDELLACTONE; DIMETHYL HYDROQUINONE; DIMETHYL METHOXY FURANONE; DIMETHYL-2,4 ACETOPHENONE; DIMETHYL-2,6 OCTANAL; DIMETHYL-3,6-BENZO-2(3H)-FURANONE; DODEC-2-EN-1,5-LACTONE; DODEC-2-EN-4-ONE; DODECANAL; E-DECALACTONE; E-DODECALACTONE; ETHYL-2-HEPTEN-2-AL; ETHYL-3 CYCLOPENTANDIONE; FARNESYL ACETONE; FURFURYLIDENE ACETONE; GAMMA DAMASCONE; GAMMA IONONE; GERANYL ACETONE; GINGERONE; HEPT-3-EN-2-ONE; HEPTANAL; HEXANAL; HEXEN 5 ONE; HEXENYL METHYL KETONE; HEXYLIDEN CYCLOPENTANONE; HOMOCORYLONE; HYDROBICYCLONE; HYDROXY-3(2)-HEPTAN-2(3)-ONE; HYDROXY-5-DECADIENOIC ACID-2,4 LACTONE; ISO JASMONE; ISOLONGIFOLENE-KETONE; ISOPHORONE; JASMIN LACTONE; LAVENDELLACTONE; MASSOIA LACTONE; MELONAL, DIMETHYL HEPTENAL; MENTHONE; METHIONAL; METHOXY-P-PHENYL-PENTEN-3-ONE, ETHONE; METHYL 2 OCTANAL; METHYL 2-PENTENE-1-AL; METHYL THIO-METHYLTHIOMETHYL-2-PENTENAL; METHYL-2 UNDECANAL; METHYL-2-BUTANAL; METHYL-2-PENT-2-ENAL; METHYL-2-TETRAHYDRO-FURANONE; METHYL-4-HYDROXY-3/2H/-FURANONE; METHYL-5-HEPT-2-EN-4-ONE; METHYL-6-HEPTA-3,5-DIENONE; METHYLTHIO-3-BUTANAL; METHYLTHIO-4-BUTAN-2-ONE; MINTLACTONE; MYRTENAL; NEOFOLIONE; NEOHESPERIDINE DIHYDROCHALCONE; NONANAL; NOOTKATONE; OCTAN-2-ONE; OCTANAL; PENNYROYAL OIL 84% PULEGONE; PENTADECANONE; PENTANAL; PENTYL 2-FURYL KETONE; PHENYL-2-BUTEN-2-AL; PHENYL-3-PROPANAL; PIPERITENONE; PIPERONYL ACETONE; P-MENTH-1-ENAL; P-MENTH-1-ENE-9-AL; P-MENTHAN-2-ONE; PRENAL; PROPANAL; PROPYL-ISO PARA ACETOPHENONE; RAC-3-HYDROXY-5-METHYL-2-(5H)-FURANONE; RASPBERRY KETONE; SAFRANAL; SAGE OIL 27% THUJONE; SOLANONE; SPIRO-(6,5)-DODECAN-1-ONE; TETRAHYDROIRONE; TETRAHYDRONOOTKATONE; TRANS-4-HEXENAL; TRANS-5-NONENAL; TRANS-6-DECEN-1-AL; TRANS-6-NONENAL; TRANS-7-METHYL-3-OCTEN-2-ONE; TRANS-7-NONENAL; TRIDECAN-2-ONE; TRIMETHYL-3,5,5-HEXANAL; TRITHIOACETONE; UNDECANAL; VANILLYLIDENE ACETONE; and VERBENONE.

3. The method according to claim 1 wherein:
R2 is selected from the group consisting of: U H, a C1 to C15 alkyl, a C1 to C15 oxoalkyl, a C1 to C15 hydroxyalkyl, C2 to C15 alkenyl, a C2 to C15 oxoalkenyl, and a C2 to C15 hydroxyalkenyl,
R3 is selected from the group consisting of: C1 to C15 straight-chain alkyl comprising one or two substituents independently selected from O, OH, N, NH, phenyl, furanyl, C9 aryl, C10 aryl, SH, and SR4, wherein R4 is a straight-chain or branched C1 to C5 alkyl residue optionally selected from methyl, ethyl, propyl, isopropyl, butyl, isobutyl, pentyl and isopentyl, and the total number of carbon atoms in R3 and R4 is up to 15, C1 to C15 straight-chain alkyl, comprising one or two atoms independently selected from O,S or N within the alkyl chain, C3 to C15 singly, doubly or multiply branched alkyl optionally substituted with one or two residues independently selected from O, OH, N, NH, SH, and SR4, wherein R4 is a straight-chain or branched C1 to C5 alkyl residue optionally selected from methyl, ethyl, propyl, isopropyl, butyl, isobutyl, pentyl and isopentyl, and the total number of carbon atoms in R3 and R4 is up to 15, C3 to C15 singly, doubly or multiply branched alkyl comprising one or two atoms independently selected from O,S or N within the alkyl chain, C2 to C15 straight-chain alkenyl, C3 to C15 straight-chain alkadienyl, C2 to C15 straight-chain alkenyl optionally substituted with one or more residues independently selected from O, OH, N, NH, phenyl, furanyl, C9 aryl, C10 aryl, SH, SR4 wherein R4 is a straight-chain or branched C1 to C5 alkenyl residue optionally selected from ethenyl, propenyl, butenyl, and pentenyl, and the total number of carbon atoms in R3 and R4 is up to 15, C2 to C15 straight-chain alkenyl, optionally comprising one or more atoms independently selected from O, S and N within the alkyl chain, C3 to C15 branched alkenyl, optionally substituted with one or two alkyl groups, C2 to C15 branched alkenyl comprising one or two alkyl groups optionally substituted with one or two residues independently selected from O, OH, N, NH, phenyl, furanyl, C9 aryl, C10 aryl, SH, SR4 wherein R4 is a straight-chain or branched C1 to C5 alkenyl residue optionally selected from ethenyl, propenyl, butenyl, and pentenyl, and the total number of carbon atoms in R3 and R4 is up to 15, C3 to C15 branched alkenyl comprising one or two alkyl groups and one or more atoms independently selected from O,S and N within the alkyl chain, C5 to C15 singly, doubly, or multiply branched alkadienyl, optionally substituted with one or two alkyl groups, C4 to C15 singly, doubly, or multiply branched alkadienyl comprising one or more atoms independently selected from O,S and N within the alkenyl chain; a 5- or 6-membered carbon ring residue comprising up to two heteroatoms independently selected from O, S, and N, optionally substituted with one or more residues independently selected from O, OH, alkoxy, alkyl, alkenyl; a C1 to C6 alkyl or alkenyl substituted with a 5- or 6-membered carbon ring residue comprising up to two heteroatoms independently selected from O, S, and N, optionally substituted with one or more residues independently selected from O, OH, alkoxy, alkyl, alkenyl; a R2-C-R3 ring residue selected from oxacyclopentan optionally substituted with a residue selected from methyl, dimethyl, ethyl, hydroxy, and methoxy, oxacyclopentene optionally substituted with a residue selected from methyl, dimethyl, ethyl, hydroxy, and methoxy, hydroxycyclopentene, hydroxyalkylcyclopentene, hydroxymethylcyclopentene, hydroxydimethylcyclopentene, ethylhydroxycyclopentene, and ethylhydroxymethylcyclopentene, thiacyclopentane, alkylthiacyclopentane, and alkyl-3-thia-cyclopentane, oxa-cyclopentene, 2-oxacyclopentene, 3-oxacyclopentene, alkyloxacylcopentene, alkyl-3-oxacyclopentene, methyl-3-oxa-cyclopentene, alkyl-3-oxacyclopentene, dimethyl-3-oxacyclopentene, ethyl-3-oxacyclopentene, ethylmethyl-3-oxacyclopentene, hydroxyalkyl-3-oxacyclopentene, hydroxymethyl-3-oxacyclopentene, hydroxydimethyl-3-oxacyclopentene, hydroxyethyl-3-oxacyclopentene, ethylhydroxymethyl-3-oxacyclopentene, oxacyclopentane, 3-oxacyclopentan, alkyl-3-oxacyclopentan, methyl-3-oxa-cyclopentan, dimethyl-3-oxacyclopentan, ethyl-3-oxacyclopentan, ethylmethyl alkyl-3-oxacyclopentan, alkylcyclopenten, alkenylcyclopenten, alkylalkenylcyclopenten, methylcylopenten, dimethylcyclopenten, ethylmethylcyclopenten, propylmethylcyclopenten, butylmethylcyclopenten, butenylmethylcyclopenten, pentylmethylcyclopenten, pentenylmethylcyclopenten, alkylcarboxy-alkenyl-cyclopentan, alkylcarboxy-alkyl-cyclopentan, methylcarboxy-alkenyl-cyclopentan, methylcarboxy-alkyl-cyclopentan, alkylcarboxy-pentenyl-cyclopentan, alkylcarboxy-pentyl-cyclopentan, methylcarboxy-alkenyl-cyclopentan, and methylcarboxy-alkyl-cyclopentan, and wherein the alkyl is selected from methyl, ethyl, propyl, butyl, pentyl, pentenyl, hexyl, heptyl, and octyl, oxo-alkylcyclohexen, oxo-methylcyclohexen, oxo-trimethylcyclohexen, oxo-ethylcyclohexen, oxo-ethylmethylcyclohexen, oxo-propylcyclohexen, oxo-propylmethylcyclohexen, oxo-isopropylcyclohexen, oxo-isopropylmethylcyclohexen, oxo-thiopropylcyclohexen, oxo-thiopropylmethylcyclohexen, alkylcyclohexen, methylcyclohexen, trimethylcyclohexen, ethylcyclohexen, ethylmethylcyclohexen, propylcyclohexen, propylmethylcyclohexen, isopropylcyclohexen, isopropylmethylcyclohexen, thiopropylcyclohexen, thiopropylmethylcyclohexen, alkylcyclohexan, methylcyclohexan, trimethylcyclohexan, ethylcyclohexan, ethylmethylcyclohexan, propylcyclohexan, propylmethylcyclohexan, isopropylcyclohexan, isopropylmethylcyclohexan, thiopropylcyclohexan, thiopropylmethylcyclohexan;
and wherein R2 and R3 together have a total number of carbon atoms of up to 15, and the R2-C-R3 has a total number of carbon atoms of up to 16.

4. The method according to claim 1 wherein R1 is an alkyl selected from the group consisting of C7, C8, C9, C10, C11, C13, C15, and C17 alkyl.

5. The method according to claim 1 wherein R1 is an alkenyl selected from the group consisting of C7, C8, C9, C10, C11, C13, C15, and C17 alkenyl, a C17-8en (oleic acid residue) alkenyl, a C17-8,11 alka-dienyl (linoleic acid residue), and a C17-8,11,14-trienyl (linolenic acid residue).

6. The method according to claim 1 wherein R2 is selected from the group consisting of H, methyl and ethyl.

7. The method according to claim 1 wherein R3 is selected from the group consisting of a C1 to C8 straight-chain alkyl, a C1 to C8 branched alkyl comprising up to two alkyl groups, wherein the alkyl residue may contain one or more further alkyl residues, a C1 to C8 straight-chain alkyl comprising one or two substituents selected from O, and SR4, wherein R4 is an alkyl residue selected from methyl, and ethyl, a C2 to C8 straight-chain alkenyl, and a ring selected from a 5 membered and a 6 membered ring, comprising up to two N heteroatoms, wherein the ring may be further substituted with one or more alkyl groups selected from methyl, ethyl, propyl and isopropyl.

8. The method according to claim 1 wherein R1 is an alkyl selected from the group consisting of C7, C9, C11, C13, C15 and C17 alkyl, R2 is selected from the group consisting of H, methyl and ethyl, and R3 is selected from the group consisting of a C1 to C8 straight-chain alkyl, a C1 to C8 branched alkyl comprising one or two alkyl groups, wherein the alkyl residue may contain one or more further alkyl residues, a C1 to C8 straight-chain alkyl comprising one or two substituents selected from O, and SR4, wherein R4 is an alkyl residue selected from methyl, and ethyl, a C2 to C8 straight-chain alkenyl, and a ring selected from a 5 membered and 6 membered ring comprising up to two N heteroatoms, wherein the ring may be further substituted with one or more alkyl groups selected from methyl, ethyl, propyl and isopropyl.

9. A flavor composition comprising at least one flavor precursor according to formula (I) 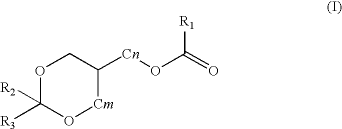
wherein n and m are selected from 0 and 1, and if n is 1 then m is 0, and if n is 0 then m is 1:
wherein R1 is selected from the group consisting of
a straight-chain C7 to C17 alkyl, a straight-chain C7 to C17 alkenyl, a branched C7 to C17 alkyl, a branched C7 to C17 alkenyl,
a straight-chain C7 to C17 monoalkenyl, a branched C7 to C17 monoalkenyl,
a straight-chain C7 to C17 alkadienyl, a branched C7 to C17 alkadienyl,
a straight-chain C7 to C17 alkatrienyl, a branched C7 to C17 alkatrienyl,
a straight-chain C9 to C17 alkatetraenyl, a branched C9 to C17 alkatetraenyl,
and a straight-chain C11 to C17 alkapentaenyl, a branched C11 to C17 alkapentaenyl, and
wherein the R2-CO-R3 part of formula I is the residue of a flavor compound, wherein the O of the R2-CO-R3 is the remainder of the reacted carbonyl group of said flavor compound,
and wherein R2-CO-R3 has a molecular weight from 44 to 294.

10. A flavor composition comprising a mixture of favor precursors according to formula (I) 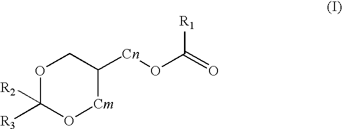
wherein n and m are selected from 0 and 1, and if n is 1 then m is 0, and if n is 0 then m is 1:
wherein R1 is selected from the group consisting of
a straight-chain C7 to C17 alkyl, a straight-chain C7 to C17 alkenyl, a branched C7 to C17 alkyl, a branched C7 to C17 alkenyl,
a straight-chain C7 to C17 monoalkenyl, a branched C7 to C17 monoalkenyl,
a straight-chain C7 to C17 alkadienyl, a branched C7 to C17 alkadienyl,
a straight-chain C7 to C17 alkatrienyl, a branched C7 to C17 alkatrienyl,
a straight-chain C9 to C17 alkatetraenyl, a branched C9 to C17 alkatetraenyl,
and a straight-chain C11 to C17 alkapentaenyl, a branched C11 to C17 alkapentaenyl, and
wherein the R2-CO-R3 part of formula I is the residue of a flavor compound, wherein the O of the R2-CO-R3 is the remainder of the reacted carbonyl group of said flavor compound,
and wherein R2-CO-R3 has a molecular weight from 44 to 294
formed by reacting at least one flavor compound comprising one or more carbonyl group with at least one monoglyceride in an acid catalyzed reaction.

11. A food product comprising at least one flavor precursor according to formula (I) 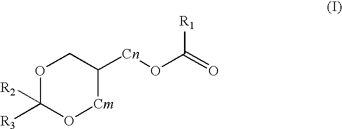
wherein n and m are selected from 0 and 1, and if n is 1 then m is 0, and if n is 0 then m is 1;
wherein R1 is selected from the group consisting of
a straight-chain C7 to C17 alkyl, a straight-chain C7 to C17 alkenyl, a branched C7 to C17 alkyl, a branched C7 to C17 alkenyl,
a straight-chain C7 to C17 monoalkenyl, a branched C7 to C17 monoalkenyl,
a straight-chain C7 to C17 alkadienyl, a branched C7 to C17 alkadienyl,
a straight-chain C7 to C17 alkatrienyl, a branched C7 to C17 alkatrienyl,
a straight-chain C9 to C17 alkatetraenyl, a branched C9 to C17 alkatetraenyl,
and a straight-chain C11 to C17 alkapentaenyl, a branched C11 to C17 alkapentaenyl, and
wherein the R2-CO-R3 part of formula I is the residue of a flavor compound, wherein the O of the R2-CO-R3 is the remainder of the reacted carbonyl group of said flavor compound,
and wherein R2-CO-R3 has a molecular weight from 44 to 294.

12. A food product comprising a mixture of flavor precursors according to formula (I) 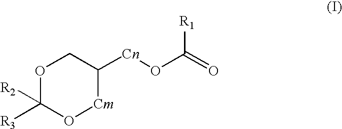
wherein n and m are selected from 0 and 1, and if n is 1 then m is 0, and if n is 0 then m is 1;
wherein R1 is selected from the group consisting of
a straight-chain C7 to C17 alkyl a straight-chain C7 to C17 alkenyl, a branched C7 to C17 alkyl, a branched C7 to C17 alkenyl.
a straight-chain C7 to C17 monoalkenyl, a branched C7 to C17 monoalkenyl,
a straight-chain C7 to C17 alkadienyl a branched C7 to C17 alkadienyl,
a straight-chain C7 to C17 alkatrienyl, a branched C7 to C17 alkatrienyl,
a straight-chain C9 to C17 alkatetraenyl, a branched C9 to C17 alkatetraenyl, and a straight-chain C11 to C17 alkapentaenyl, a branched C11 to C17 alkapentaenyl and
wherein the R2-CO-R3 part of formula I is the residue of a flavor compound, wherein the O of the R2-CO-R3 is the remainder of the reacted carbonyl group of said flavor compound,
and wherein R2-CO-R3 has a molecular weight from 44 to 294.
formed by reacting at least one flavor compound comprising one or more carbonyl group with at least one monoglyceride in an acid catalyzed reaction.

13. A flavor precursor compound according to formula (I) 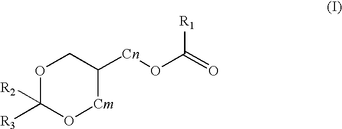
wherein n=0 and m=1;
wherein R1 is selected from the group consisting of
a straight-chain C7 to C17 alkyl, a straight-chain C7 to C17 alkenyl, a branched C7 to C17 alkyl, a branched C7 to C17 alkenyl,
a straight-chain C7 to C17 monoalkenyl, a branched C7 to C17 monoalkenyl,
a straight-chain C7 to C17 alkadienyl, a branched C7 to C17 alkadienyl,
a straight-chain C7 to C17 alkatrienyl, a branched C7 to C17 alkatrienyl
a straight-chain C9 to C17 alkatetraenyl, a branched C9 to C17 alkatetraenyl,
and a straight-chain C11 to C17 alkapentaenyl, a branched C11 to C17 alkapentaenyl, and
wherein the R2-CO-R3 part of formula I is the residue of a flavor compound, wherein the O of the R2-CO-R3 is the remainder of the reacted carbonyl group of said flavor compound,
and wherein R2-CO-R3 has a molecular weight from 44 to 294.
wherein m=1 and n=0.

14. A process of producing the flavor precursor compound of claim 13 comprising the step of: reacting at least one flavor compound comprising one or more carbonyl group with at least one monoglyceride in an acid catalyzed reaction.

15. (canceled)

16. (canceled)
